(19) United States
(12) Patent Application Publication
(10) Pub. No.: US 2006/0240443 A1
(43) Pub. Date: Oct. 26, 2006

(54) Microarray-based single nucleotide polymorphism, sequencing, and gene expression assay method

(75) Inventors: Michael J. Lodes, Seattle, WA (US); Dominic Suciu, Edmonds, WA (US); Andy McShea, Kingston, WA (US)

Correspondence Address: COMBIMATRIX CORPORATION, 6500 HARBOUR HEIGHTS PARKWAY, MUKILTEO, WA, 98275, US

(73) Assignee: CombiMatrix Corporation

(21) Appl. No.: 11/110,630

(22) Filed: Apr. 20, 2005

(51) Int. Cl. C12Q 1/68; C12P 19/34

(52) U.S. Cl. 435/6; 435/91.2

(57) ABSTRACT

There is disclosed a microarray-based single nucleotide polymorphism, sequencing, and gene expression assay method. Specifically, there is disclosed a method using a microarray device wherein a plurality of hybridized structures is formed by contacting the microarray under a hybridizing condition to a hybridizing solution comprising a plurality of tagged targets and a plurality of detection sequences. The detection sequences of each hybridized structure is extended using an extension-ligation solution and an extension-ligation condition. After extension, ligation of the extended sequence occurs to a probe if the terminal nucleotide of a probe is complementary to the hybridized tagged targets. Non-bound material is removed by using a washing solution and a washing method. The target nucleotide and the target sequence of the tagged targets is determined by which probe is ligated to the detection sequences.

### TECHNICAL FIELD OF THE INVENTION

The present invention provides a method for determining single nucleotide polymorphisms (SNPs), sequencing a gene or a sequence of interest, and for gene expression, each using a microarray device. More particularly, the present invention provides to a method for hybridization, extension, and ligation of nucleic acid sequences on a microarray device.

### BACKGROUND OF THE INVENTION

Microarrays have become important analytical research tools in pharmacological and biochemical research and discovery. Microarrays are miniaturized arrays of points on a solid surface. The surface is sometimes planar. Molecules, including biomolecules, may be attached or synthesized in situ at specific attachment points on a microarray. The attachment points are usually in a column and row format although other formats may be used. An advantage of microarrays is that they provide the ability to conduct hundreds, if not thousands, of experiments in parallel. Such parallelism, as compared to sequential experimentation, can be used to increase the efficiency of exploring relationships between molecular structure and biological function, where slight variations in chemical structure can have profound biochemical effects. Microarrays are available in different formats and have different surface chemistry characteristics. The differences result in different approaches for attaching or synthesizing molecules on a microarray. Differences in surface chemistry lead to differences in preparation methods for providing a surface that is receptive to attachment of a pre-synthesized chemical species or for synthesizing a chemical species in situ. As the name suggests, the attachment points on microarrays are of a micrometer scale, which is generally 1-100 μm.

Research using microarrays has focused mainly on deoxyribonucleic acid (DNA) and ribonucleic acid (RNA) related areas, which includes genomics, cellular gene expression, single nucleotide polymorphisms (SNP), genomic DNA detection and validation, functional genomics, and proteomics (Wilgenbus and Lichter, J. Mol. Med. 77:761, 1999; Ashfari et al., Cancer Res. 59:4759, 1999; Kurian et al., J. Pathol. 187:267, 1999; Hacia, Nature Genetics 21 suppl.:42, 1999; Hacia et al., Mol. Psychiatry 3:483, 1998; and Johnson, Curr. Biol. 26:R171, 1998.) In addition to microarrays for DNA/RNA research, microarrays can be used for research related to peptides (two or more linked natural or synthetic amino acids), small molecules (such as pharmaceutical compounds), oligomers, and polymers.

There are numerous methods for preparing a microarray of DNA related molecules. DNA related molecules include native or cloned DNA and synthetic DNA. Synthetic, relatively short single-stranded DNA or RNA strands are commonly referred to as oligonucleotides (oligos), which is synonymous with oligodeoxyribonucleotide. Microarray preparation methods include the following: (1) spotting a solution on a prepared flat surface using spotting robots; (2) in situ synthesis by printing reagents via ink jet or other printing technology and using regular phosphoramidite chemistry; (3) in situ parallel synthesis using electrochemically generated acid for deprotection and using regular phosphoramidite chemistry; (4) maskless photo-generated acid (PGA) controlled in situ synthesis and using regular phosphoramidite chemistry; (5) mask-directed in situ parallel synthesis using photo-cleavage of photolabile protecting groups (PLPG); (6) maskless in situ parallel synthesis using PLPG and digital photolithography; and (7) electric field attraction/repulsion for depositing oligos.

Photolithographic techniques for in situ oligo synthesis are disclosed in Fodor et al. U.S. Pat. No. 5,445,934 and the additional patents claiming priority thereto. Electric field attraction/repulsion microarrays are disclosed in Hollis et al. U.S. Pat. No. 5,653,939 and Heller et al. U.S. Pat. No. 5,929,208. An electrode microarray for in situ oligo synthesis using electrochemical deblocking is disclosed in Montgomery, U.S. Pat. Nos. 6,093,302, 6,280,595, and 6,444,111 (Montgomery I, II, and III respectively), which are incorporated by reference herein. A different array (not a microarray) having parallel rows of linear electrodes for in situ oligo synthesis at adjacent surfaces but not on the array but using electrochemical deblocking is disclosed in Southern, U.S. Pat. No. 5,667,667. A review of oligo microarray synthesis is provided by: Gao et al., Biopolymers 73:579, 2004.

The electrochemical synthesis microarray disclosed in Montgomery I, II, and III is based upon a semiconductor chip having a plurality of microelectrodes in a column and row format. This chip design uses Complementary Metal Oxide Semiconductor (CMOS) technology to create high-density arrays of microelectrodes with parallel addressing for selecting and controlling individual microelectrodes within the array. In order to provide appropriate reactive groups at each electrode, the microarray is coated with a porous matrix material. Biomolecules as well as other molecules can be synthesized at any of the electrodes on the porous matrix. The electrodes are “turned on” by applying a voltage or current that generates electrochemical reagents (particularly acidic protons) that alter the pH in a small, defined “virtual flask” region or volume adjacent to the electrode. The electrochemically-generated reagents remove protective groups to allow continued synthesis of a DNA or other oligomeric or polymeric material. The pH decreases only in the vicinity of the electrode because the ability of the acidic reagent to travel away from an electrode is limited by natural diffusion and by buffering.

The problem of identifying single nucleotide polymorphisms (SNPs) may be addressed by using microarrays. Considering that there are over 10 million SNPs estimated to occur in the human genome, microarrays may provide an opportunity to more quickly identify SNPs. (The International HapMap Consortium, The International HapMap Project, Nature, 426:789-796, 2003). Many SNPs have been associated directly or indirectly with genetic diseases including Crohn's disease, ataxia telangiectasia, and Alzheimer's disease.

Considering Crohn's disease for example, some patients have been shown to have genetic mutations in one or more of several genes that are associated with increased susceptibility to the disease. Such associated genes include the CARD15/NOD2 gene and the MDR1 gene (Helio et al., Gut 52:558-562 2003; Newman et al., Am. J. Gastroenterol. 99:306-315 2004; and Brant et al., Am. J. Hum. Genet. 73:1282-1292, 2003).

Moreover, mutations in the ataxia telangiectasia mutated (ATM) gene have been shown to be associated with lymphoma and ataxia telangiectasia, which is characterized by cerebellar and neuromotor degeneration and immune deficiency (Fang et al., Proc. Natl. Acad. Sci. USA 100:5372-5377, 2003; and Hacia et al., Genome Research 8:1245-1258 1998).

The ability to detect mutations in genomes allows a more specific diagnosis and therapy as well as prediction of whether a person may be prone to a genetically related disease. Such prediction can be especially important when there is a family history of genetic disease. Detection of genetic mutations (i.e., a propensity for a genetic disease) has been approached using several technologies for detecting SNPs. Such technologies include (1) oligonucleotide microarray-based hybridization methods, (2) enzymatic methods, and (3) mass spectrometry (Kirk et al., Nucleic Acids Res. 30:3295-3311 2002; Kwok, Annu. Rev. Genomics Hum. Genet. 2:235-258, 2001; Jenkins & Gibson, Comp. Funct. Genom. 3:57-66 2002; and Shi, Clinical Chemistry 47:164-172 2001).

Oligonucleotide microarray-based hybridization methods of SNP have been used to screen for both previously characterized SNP and for the discovery of SNP (Hacia, Nature Genetics Supplement 21:42-47 1999). In the microarray hybridization method, either a gain of signal method or a loss signal method is used. The signal comes from a fluorescent tag attached to a particular probe. In the gain of signal method, oligonucleotide probes having a complementary portion to sequence changes of interest. Gain of hybridization signal for the probes is measured relative to reference samples. In the loss of hybridization signal method, loss of hybridization signal is analyzed using perfect match probes complementary to the wild-type sequence. Loss of hybridization signal for the probes is measured relative to reference samples.

There are inherent difficulties with the hybridization method that limit its ability to accurately detect SNPs. Such limitations include, for example, the inadequate ability (1) to detect the difference between heterozygous base changes compared to homozygous mutations, (2) to detect intramolecular and intermolecular structures such as hairpin and G-rich sequences, and (3) to simultaneously locate SNPs in G/C-rich sequences and A/T-rich sequences due to differences in melting temperature of such sequences. Differences in melting temperature cause either sub-optimal hybridization conditions for G/C-rich sequences must be used to detect A/T-rich sequences or cause A/T-rich probes to have to be increased in length to equalize hybridization conditions. Increasing length reduces the ability to detect SNP in A/T-rich sequences. As a result of high hybridization stringency, the hybridization method provides high accuracy on only 65% of the DNA surveyed (Patil et al., Science, 294:1719-1723 2001).

Enzymatic methods (nucleotide extension, cleavage, or ligation) have also been used for SNP detection (mismatch discrimination). These procedures include primer extension or mini-sequencing and ligation of probes to sequence specific primers using a genomic sequence as a hybridization template (see, for example, Broude et al., Proc. Natl. Acad. Sci. USA 91:3072-3076 1994; Dubiley et al., Nucleic Acids Research 25:2259-2265 1997; O'Meara et al., Nucleic Acids Research 30:e75, 1-8 2002; and Rickert et al., Analytical Biochemistry 330:288-297 2004). Primer extension, or mini-sequencing is a technique that involves the extension on single-stranded amplified genomic DNA of a specific primer in the presence of polymerase and either fluorescent ddNTPs or 1 ddNTP and 3 dNTPs. Detection is accomplished with gel or capillary sequencing or MALDI/TOF (Kirk et al., Nucleic Acids Research 30:3295-3311 2002).

Ligation reactions generally require two adjacent primers to anneal to a genome-derived target. The upstream primer generally contains a label on the 5′ end. The 3′ nucleotide is designed to be opposite the SNP of interest. When the 3′ nucleotide forms a perfect match with the target, the primer (with label) is covalently attached by ligase to the downstream primer. Detection is by fluorescent display on a microarray or by MALDI/TOF (Kirk et al., Nucleic Acids Research 30:3295-3311 2002; Zhong et al., Proc. Natl. Acad. Sci. USA 100:11559-11564 2003; Iannone et al., Cytometry 39:131-140 2000; Chen et al., Genome Research 8:449-556 1998; and Consolandi et al., Hum Mutat. 24:428-434 2004). One disadvantage in this procedure is the expense of using labeled, specific primers for the SNPs being screened.

The present invention overcomes the limitations of microarray SNP hybridization methods and enzyme methods by providing a SNP, sequencing, and gene expression assay method on a microarray device. The inventive method combines the sensitivity and specificity of ligation with the cost effective strategy of using a labeled common oligonucleotide.

### SUMMARY OF THE INVENTION

The present invention provides a microarray-based single nucleotide polymorphism, sequencing, and gene expression assay method. In inventive method comprises (1) providing a microarray device having a plurality of oligonucleotide probes attached thereto, wherein each probe has a terminal nucleotide that is complementary to a target nucleotide; (2) forming a plurality of hybridized structures on the microarray, wherein each hybridized structure is formed by contacting the microarray under a hybridizing condition to a hybridizing solution comprising a plurality of tagged targets and a plurality of detection sequences, wherein each hybridized structure comprises one tagged target hybridized to one probe and to one detection sequence; (3) extending each hybridized structure using an extension-ligation solution; (4) removing non-bound material by washing the microarray; and (5) identifying the target nucleotide and a hybridized sequence from the hybridized structures having ligation.

Preferably, the microarray device having a plurality of oligonucleotide probes attached thereto is made by a method selected from the group consisting of spotting oligonucleotides directly on the microarray by various computer printing techniques (e.g., ink jet printing) and synthesizing each oligonucleotide in situ on the microarray. More preferably, the microarray is an electrode array device, wherein the plurality of probes is synthesized in situ on the electrode microarray using an electrochemical technique. Preferably, the plurality of probes is selected from the group consisting of probe DNA and probe RNA, and combinations thereof. Preferably, the plurality of probes is attached to the microarray by a spacer.

The plurality of tagged targets is selected from the group consisting of tagged target DNA and tagged target RNA, and combinations thereof. The tagged target DNA may be a cDNA. The tagged target RNA may be an mRNA. The plurality of tagged targets may be first amplified. Preferably, the amplification is by PCR.

The plurality of detection sequences is selected from the group consisting of a detection sequence DNA and a detection sequence RNA, and combinations thereof. Preferably, the plurality of detection sequences has a fluorescent tag.

Preferably, the plurality of tagged targets and the plurality of probes have less than approximately five internal mismatches when hybridized. Preferably, the plurality of tagged targets and the plurality of detection sequences have less than about five internal mismatches when hybridized. Preferably, the hybridizing solution comprises a plurality of tagged targets and a plurality of detection sequences in a buffer solution comprising a 1×T4 ligase buffer. Preferably, the hybridizing condition comprises approximately 45° C. for approximately one hour. Preferably, the extension-ligation solution comprises water, buffer, triphosphate mix, polymerase, and ligase. Preferably, the extension-ligation condition comprises incubation of the microarray exposed to the extension-ligation solution at approximately thirty-seven degrees centigrade for approximately one hour.

The polymerase is selected from the group consisting of DNA polymerase and RNA polymerase, and combinations thereof. Preferably, the polymerase is selected from the group consisting of Taq polymerase Stoffel fragment, a reverse transcriptase, E. coli DNA polymerase, Klenow fragment polymerase, T7 RNA polymerase, T3 RNA polymerase, viral replicase, SP6 RNA polymerase, and combinations thereof. Preferably, the buffer is selected from the group consisting of T4 DNA ligase buffer and T4 RNA ligase buffer, and combinations thereof. Preferably, the ligase is selected from the group consisting of E. coli DNA ligase, T4 DNA ligase, and T4 RNA ligase, and combinations thereof. Preferably, the triphosphate mix is selected from the group consisting of dNTP and rNTP.

Preferably, the wash solution is selected from the group consisting of buffer solution and base solution. Preferably, the buffer is a Tris buffer or a phosphate buffer. Preferably, the base solution is an aqueous sodium hydroxide solution. Preferably, the wash method comprises exposing the microarray to the wash solution at a temperature of approximately room temperature to approximately seventy degrees centigrade.

The present invention further provides a microarray-based single nucleotide polymorphism, sequencing and gene expression assay method comprising (1) providing a microarray device having a plurality of oligonucleotide probe sequences at defined locations thereon; (2) forming a plurality of hybridized structure DNA's wherein each hybridized structure DNA is formed by contacting the microarray device under hybridizing conditions to a hybridizing solution comprising a plurality of tagged target DNA sequences and a plurality of detection sequence DNAs, wherein each hybridized structure DNA comprises one tagged target DNA hybridized to one oligonucleotide probe DNA and to one detection sequence DNA; (3) extending each hybridized structure DNA using an extension-ligation solution and an extension-ligation condition; (4) ligating each hybridized structure DNA having a terminal nucleotide DNA that is complementary to a target nucleotide DNA using the extension-ligation solution and the extension-ligation condition; (4) removing non-bound material by washing the microarray device using a wash solution; and (5) identifying the target nucleotide DNA and a hybridized sequence DNA from the hybridized structures having ligation.

Preferably, the microarray device having a plurality of oligonucleotide probes attached thereto is made by a method selected from the group consisting of spotting oligonucleotides directly on the microarray by various computer printing techniques (e.g., ink jet printing) and synthesizing each oligonucleotide in situ on the microarray. Preferably, the plurality of probe DNA is attached to the microarray by a spacer. Preferably, the microarray is an electrode microarray, wherein the plurality of probes is synthesized in situ on the electrode microarray. The tagged target DNA may be a cDNA. The tagged target DNA may be first amplified. Preferably, the amplification is by PCR. Preferably, the plurality of detection sequence DNA has a fluorescent tag. Preferably, the plurality of tagged target DNA and the plurality of probe DNA have less than five internal mismatches when hybridized. Preferably, the plurality of tagged target DNA and the plurality of detection sequence DNA has less than approximately five internal mismatches when hybridized.

Preferably, the hybridizing solution comprises a plurality of tagged target DNA and a plurality of detection sequence DNA in a buffer solution comprising a 1×T4 ligase buffer. Preferably, the hybridizing condition comprises approximately 45° C. for approximately one hour. Preferably, the extension-ligation solution comprises water, buffer, dNTP, polymerase, and ligase. Preferably, the extension-ligation condition comprises incubation of the microarray exposed to the extension-ligation solution at approximately thirty-seven degrees centigrade for approximately one hour. The polymerase is a DNA polymerase. Preferably, the DNA polymerase is selected from the group consisting of Taq polymerase Stoffel fragment, a reverse transcriptase, E. coli polymerase, and, Klenow fragment polymerase, and combinations thereof.

Preferably, the buffer comprises E. coli ligase buffer, and the ligase comprises E. coli ligase. Alternatively, the buffer comprises T4 ligase buffer and the ligase comprises T4 DNA ligase. Preferably, the wash solution is selected from the group consisting of buffer solution and base solution. Preferably, the buffer is a Tris buffer or a phosphate buffer. Preferably, the base solution is an aqueous sodium hydroxide solution. Preferably, the wash method comprises exposing the microarray to the wash solution at a temperature of approximately room temperature to approximately seventy degrees centigrade.

In an alternative embodiment, the plurality of probe DNA comprises a plurality of match probe DNA and a plurality of mismatch probe DNA, and the plurality of hybridized structure DNA comprises a plurality of match structures and a plurality of mismatch structures. In an alternative embodiment, the plurality of probe DNA comprises a plurality of set probes, and the plurality of hybridized structure DNA comprises a plurality of set structures. In an alternative embodiment, the plurality of probe DNA comprises a plurality of consecutive sequence probes, and the plurality of hybridized structure DNA comprises a plurality of consecutive sequence structures. In an alternative embodiment, the plurality of probe DNA comprises a plurality of gene expression probes, and the plurality of hybridized structure DNA comprises a plurality of gene expression structures.

The present invention further provides a method for a microarray-based single nucleotide polymorphism, sequencing, and gene expression assay method comprising (1) providing a microarray device having a plurality of oligonucleotide probe DNA sequences; (2) forming a plurality of hybridized structure DNA/RNA on the microarray device, wherein each hybridized structure DNA/RNA is formed by contacting the microarray device under a hybridizing conditions to a hybridizing solution comprising a plurality of tagged target RNA and a plurality of detection sequence DNA, wherein each hybridized structure DNA/RNA comprises one tagged target RNA hybridized to one probe DNA and to one detection sequence DNA; (3) extending each hybridized structure DNA/RNA using an extension-ligation solution and an extension-ligation condition; (4) ligating each hybridized structure DNA/RNA having a terminal nucleotide DNA that is complementary to a target nucleotide RNA using the extension-ligation solution; (5) removing non-bound material by washing the microarray device using a wash solution; and (6) identifying the target nucleotide RNA and a hybridized sequence RNA from the hybridized structures having ligation.

Preferably, the microarray device having a plurality of oligonucleotide probes attached thereto is made by a method selected from the group consisting of spotting oligonucleotides directly on the microarray by various computer printing techniques (e.g., ink jet printing) and synthesizing each oligonucleotide in situ on the microarray. Preferably, the plurality of probes is attached to the microarray by a spacer. Preferably, the microarray is an electrode microarray, wherein the plurality of probes are synthesized in situ on the electrode microarray. The tagged target RNA may be an mRNA. Preferably, the plurality of detection sequence DNA has a fluorescent tag. Preferably, the plurality of tagged target RNA and the plurality of probe DNA have less than five internal mismatches when hybridized. Preferably, the plurality of tagged target RNA and the plurality of detection sequence DNA has less than approximately five internal mismatches when hybridized.

Preferably, the hybridizing solution comprises a plurality of tagged target RNA and a plurality of detection sequence DNA in a buffer solution comprising a 1×T4 ligase buffer, and the hybridizing condition comprises approximately 45° C. for approximately one hour.

Preferably, the extension-ligation solution comprises water, buffer, dNTP, polymerase, and ligase. Preferably, the extension-ligation condition comprises incubation of the microarray exposed to the extension-ligation solution at approximately thirty-seven degrees centigrade for approximately one hour. Preferably, the polymerase is a reverse transcriptase. Preferably, the buffer comprises E. coli ligase buffer, and the ligase comprises E. coli ligase. Alternatively, the buffer comprises T4 ligase buffer and the ligase comprises T4 DNA ligase. Preferably, the wash solution is selected from the group consisting of buffer solution and base solution. Preferably, the buffer is a Tris buffer or a phosphate buffer. Preferably, the base solution is an aqueous sodium hydroxide solution. Preferably, the wash method comprises exposing the microarray to the wash solution at a temperature of approximately room temperature to approximately seventy degrees centigrade.

### BRIEF DESCRIPTION OF THE DRAWINGS

FIGS. 1A-1F show the sequence of steps for an inventive SNP assay method. Specifically, FIG. 1A is a schematic of a microarray prior to addition of probe DNA. FIG. 1B is a schematic of the microarray having match probe DNA and mismatch probe DNA. FIG. 1C is a schematic of the microarray having tagged target DNA and detection sequence DNA hybridized to each probe to form hybridized structure DNA. FIG. 1D is a schematic showing ligation when the terminal nucleotide DNA is complementary to the target nucleotide DNA. FIG. 1E is a schematic showing the microarray after washing off the unligated detection sequence DNA and the tagged target DNA. FIG. 1F is a schematic showing a detectable signal, such as a fluorescent signal, at microarray locations having the detection sequence DNA ligated to probe DNA.

FIGS. 2A and 2B are schematics showing the PCR amplification method and isolation of tagged target DNA from genomic DNA having target DNA.

FIG. 3 is a schematic of a tagged target, a detection sequence having a fluorescent (Cy3) label, and a probe.

FIGS. 4A and 4B are schematics showing an inventive SNP method where there are four identical probes having different terminal nucleotides.

FIG. 5 is a schematic showing sequencing of a DNA strand to locate a SNP or mutation along the strand by designing probes for each location on the strand.

FIG. 6 is a bar chart comparison of the microarray-based SNP assay of the present invention compared to SNP detection using hybridization to detect an internal SNP. The data is of the ATM gene of Patient #4, who has no mutations. Genomic DNA from Patient #4 with no symptoms of ataxia was amplified with four primer sets designed to isolate the areas of four known SNP. Probes on the microarray device were designed so that the predicted melting temperature (TM) was approximately 50° C.

FIG. 7 is an expanded view of the bar chart comparison as shown in FIG. 6. FIG. 7 compares the terminal SNP assay of the present invention to internal SNP hybridization. A ten nucleotide spacer was use to attach the probe DNA to the electrode microarray. The wild-type or unmodified is the first bar, the SNP is the second bar, and the last two bars are non-sense mutations.

FIG. 8 is a bar chart comparing probe DNA adjusted for a constant melting temperature (TM; left panel) and shows an inverse relationship between percentages of G/C content (right panel) and probe DNA length (numbers on bars). Thus, a probe DNA with a high G/C content will be short (15 nucleotides for internal 7327) while a probe DNA with a high A/T content will be relatively longer (28 nucleotides for internal 8266.) Under certain hybridization conditions, the longer probe DNA will not discriminate a mismatch with hybridization alone.

FIG. 9 is a bar chart showing the results of a multiplex SNP assay (four tagged target DNA's) on genomic DNA from Patient #1 having an ATM gene SNP at residue 103C>T. The vertical bars indicate the mean of eight replicates, and the vertical lines indicate plus or minus one standard deviation. The first probe DNA in each series of four probes is wild type. The second probe DNA is the SNP. The third probe DNA and fourth probe DNA are non-sense mutations. Also shown are the results of the inclusion of a 5, 10, or 15 nucleotide spacer between the electrode microarray and the sequence of interest. The probes representing sequences of interest for each SNP have been boxed below, and the terminal nucleotide for each probe is shown above each graph.

FIG. 10 is a bar chart showing the results of a multiplex SNP assay (four tagged target DNA's) on genomic DNA from Patient #2 having an ATM gene SNP at residues 7327C>T and 7926A>C. The vertical bars indicate the mean of eight replicates, and the vertical lines indicate plus or minus one standard deviation. The first probe DNA in each series of four probes is wild type. The second probe DNA is the SNP. The third probe DNA and fourth probe DNA are non-sense mutations. Also shown are the results of the inclusion of a 5, 10, or 15 nucleotide spacer between the electrode microarray and the sequence of interest. The probes representing sequences of interest for each SNP have been boxed below, and the terminal nucleotide for each probe is shown above each graph.

FIG. 11 is a bar chart showing the results of a multiplex SNP assay (four tagged target DNA's) on genomic DNA from Patient #3 having an ATM gene SNP at residue 8266A>T. The vertical bars indicate the mean of eight replicates, and the vertical lines indicate plus or minus one standard deviation. The first probe DNA in each series of four probes is wild type. The second probe DNA is the SNP. The third probe DNA and fourth probe DNA are non-sense mutations. Also shown are the results of the inclusion of a 5, 10, or 15 nucleotide spacer between the electrode microarray and the sequence of interest. The probes representing sequences of interest for each SNP have been boxed below, and the terminal nucleotide for each probe is shown above each graph.

FIG. 12 is a bar chart showing the results of a multiplex SNP assay (four tagged target DNA's) on genomic DNA from Patient #4 having no ATM gene SNP residues. The vertical bars indicate the mean of eight replicates, and the vertical lines indicate plus or minus one standard deviation. The first probe DNA in each series of four probes is wild type. The second probe DNA is the SNP. The third probe DNA and fourth probe DNA are non-sense mutations. Also shown are the results of the inclusion of a 5, 10, or 15 nucleotide spacer between the electrode microarray and the sequence of interest.

### DETAILED DESCRIPTION OF THE INVENTION

Definitions

As used herein, the term “oligomer” means a molecule of intermediate relative molecular mass, the structure of which essentially comprises a small plurality of units derived, actually or conceptually, from molecules of lower relative molecular mass. A molecule is regarded as having an intermediate relative molecular mass if it has properties which do vary significantly with the removal of one or a few of the units. If a part or the whole of the molecule has an intermediate relative molecular mass and essentially comprises a small plurality of units derived, actually or conceptually, from molecules of lower relative molecular mass, it may be described as oligomeric, or by oligomer used adjectivally. Oligomers are typically comprised of one type of monomer (mer.) A preferred oligomer is an oligonucleotide.

The term “co-oligomer” means an oligomer derived from more than one species of monomer. The term oligomer includes co-oligomers. As examples of oligomers, a single stranded DNA molecule consisting of deoxyadenylate (A), deoxycytidylate (C), deoxyguanylate (G), and deoxythymidylate (T) units in the following sequence, AGCTGCTATA is a co-oligomer, and a single stranded DNA molecule consisting of 10-T units is an oligomer; however, both are referred to as oligomers.

The term “monomer” or “mer” means a molecule that can undergo polymerization thereby contributing constitutional units to the essential structure of a macromolecule such as an oligomer, co-oligomer, polymer, or co-polymer. Examples of monomers include A, C, G, T/U, adenylate, guanylate, cytidylate, uridylate, amino acids, vinyl chloride, and other vinyls.

The term “polymer” means a substance composed of macromolecules, which is a molecule of high relative molecular mass, the structure of which essentially comprises the multiple repetition of units derived, actually or conceptually, from molecules of low relative molecular mass. In many cases, especially for synthetic polymers, a molecule can be regarded as having a high relative molecular mass if the addition or removal of one or a few of the units has a negligible effect on the molecular properties. This statement fails in the case of certain macromolecules for which the properties may be critically dependent on fine details of the molecular structure. If a part or the whole of the molecule has a high relative molecular mass and essentially comprises multiple repetition of units derived, actually or conceptually, from molecules of low relative molecular mass, it may be described as either macromolecular or polymeric, or by polymer used adjectivally.

The term “copolymer” means a polymer derived from more than one species of monomer. Copolymers that are obtained by copolymerization of two monomer species are sometimes termed bipolymers, those obtained from three monomers terpolymers, those obtained from four monomers quaterpolymers, etc. The term polymer includes co-polymers.

The term “polyethylene glycol” (PEG) means an organic chemical having a chain consisting of the common repeating ethylene glycol unit [—CH₂—CH₂—O—]ₙ. PEG's are typically long chain organic polymers that are flexible, hydrophilic, enzymatically stable, and biologically inert, but they do not have an ionic charge in water. In general, PEG can be divided into two categories. First, there is polymeric PEG having a molecular weight ranging from 1000 to greater than 20,000. Second, there are PEG-like chains having a molecular weight that is less than 1000. Polymeric PEG has been used in bioconjugates, and numerous reviews have described the attachment of this linker moiety to various molecules. PEG has been used as a linker, where the short PEG-like linkers can be classified into two types, the homo-[X—(CH₂—CH₂—O)ₙ]—X and heterobifunctional [X—(CH₂—CH₂—O)ₙ]—Y spacers.

The term “PEG derivative” means an ethylene glycol derivative having the common repeating unit of PEG. Examples of PEG derivatives include diethylene glycol (DEG), tetraethylene glycol (TEG), polyethylene glycol having primary amino groups, di(ethylene glycol) mono allyl ether, di(ethylene glycol)mono tosylate, tri(ethylene glycol)mono allyl ether, tri(ethylene glycol)mono tosylate, tri(ethylene glycol)mono benzyl ether, tri(ethylene glycol) mono trityl ether, tri(ethylene glycol)mono chloro mono methyl ether, tri(ethylene glycol)mono tosyl mono allyl ether, tri(ethylene glycol)mono allyl mono methyl ether, tetra(ethlyne glycol) mono allyl ether, tetra(ethylene glycol)mono methyl ether, tetra(ethylene glycol)mono tosyl mono allyl ether, tetra(ethylene glycol)mono tosylate, tetra(ethylene glycol)mono benzyl ether, tetra(ethylene glycol)mono trityl ether, tetra(ethylene glycol)mono 1-hexenyl ether, tetra(ethylene glycol)mono 1-heptenyl ether, tetra(ethylene glycol)mono 1-octenyl ether, tetra(ethylene glycol)mono 1-decenyl ether, tetra(ethylene glycol)mono 1-undecenyl ether, penta(ethylene glycol)mono methyl ether, penta(ethylene glycol)mono allyl mono methyl ether, penta(ethylene glycol)mono tosyl mono methyl ether, penta(ethylene glycol)mono tosyl mono allyl ether, hexa(ethylene glycol)mono allyl ether, hexa(ethylene glycol)mono methyl ether, hexa(ethylene glycol)mono benzyl ether, hexa(ethylene glycol)mono trityl ether, hexa(ethylene glycol)mono 1-hexenyl ether, hexa(ethylene glycol)mono 1-heptenyl ether, hexa(ethylene glycol) mono 1-octenyl ether, hexa(ethylene glycol)mono 1-decenyl ether, hexa(ethylene glycol)mono 1-undecenyl ether, hexa(ethylene glycol)mono 4-benzophenonyl mono 1-undecenyl ether, hepta(ethylene glycol)mono allyl ether, hepta(ethylene glycol)mono methyl ether, hepta(ethylene glycol)mono tosyl mono methyl ether, hepta(ethylene glycol)monoallyl mono methyl ether, octa(ethylene glycol)mono allyl ether, octa(ethylene glycol)mono tosylate, octa(ethylene glycol) mono tosyl mono allyl ether, undeca(ethylene glycol)mono methyl ether, undeca(ethylene glycol) mono allyl mono methyl ether, undeca(ethylene glycol)mono tosyl mono methyl ether, undeca(ethylene glycol)mono allyl ether, octadeca(ethylene glycol)mono allyl ether, octa(ethylene glycol), deca(ethylene glycol), dodeca(ethylene glycol), tetradeca(ethylene glycol), hexadeca(ethylene glycol), octadeca(ethylene glycol), benzophenone-4-hexa(ethylene glycol)allyl ether, benzophenone-4-hexa(ethylene glycol)hexenyl ether, benzophenone-4-hexa(ethylene glycol)octenyl ether, benzophenone-4-hexa(ethylene glycol)decenyl ether, benzophenone-4-hexa(ethylene glycol)undecenyl ether, 4-flourobenzophenone-4′-hexa(ethylene glycol)allyl ether, 4-flourobenzophenone-4-hexa(ethylene glycol)undecenyl ether, 4-hydroxybenzophenone-4-hexa(ethylene glycol)allyl ether, 4-hydroxybenzophenone-4′-hexa(ethylene glycol)undecenyl ether, 4-hydroxybenzophenone-4-tetra(ethylene glycol)allyl ether, 4-hydroxybenzophenone-4-tetra(ethylene glycol)undecenyl ether, 4-morpholinobenzophenone-4′-hexa(ethylene glycol)allyl ether, 4-morpholinobenzophenone-4′-hexa(ethylene glycol)undecenyl ether, 4-morpholinobenzophenone-4-tetra(ethylene glycol)allyl ether, and 4-morpholinobenzophenone-4′-tetra(ethylene glycol)undecenyl ether.

The term “single nucleotide polymorphism” (SNP) means substitution of a deoxynucleotide in a single stranded DNA sequence by a different deoxynucleotide. Such substitution is commonly referred to as single base substitution, where the bases are adenine, guanine, cytosine, and thymine. The respective deoxynucleotides are deoxyadenylate, deoxyguanylate, deoxycytidylate, and deoxythymidylate. The bases and the deoxynucleotides are represented as A, G, C, and T respectively. When a SNP is linked to a specific genetic disorder, the deoxynucleotide present before substitution is considered the normal or wild type deoxynucleotide, and the deoxynucleotide that is substituted is considered the SNP or the mutation. For example, if the DNA sequence atatgcact [SEQ ID NO:1] is considered normal, where the first A is numbered 1 in the gene sequence, then the DNA sequence atattcact [SEQ ID NO:2] is considered to have a G to T substitution at the fifth location on the gene sequence. The term also includes mutations on an RNA chain such as a viral RNA. Copy DNA (cDNA) may be used to represent an RNA chain and any accompanying mutations. cDNA is made from the RNA of interest using a reverse transcriptase enzyme.

The term “wild type” means the form of an organism, as it is predominately found in nature in contrast to domesticated strains, natural mutations, or laboratory mutations. For example, a portion of a wild type gene could be atatgccgt [SEQ ID NO:3], and a 4T>C “mutation” would be atacgccgt [SEQ ID NO:4]. The terms wild type and mutation encompass DNA and RNA based organisms.

The term “microarray” means a solid substrate having locations thereon for placing DNA or other chemical species. Placing includes in situ synthesis and spotting of pre-synthesized materials. The term includes an electrode microarray, wherein the locations are electrodes. Single stranded DNA or RNA or other chemical species can be synthesized in situ on individual electrodes. Microarrays are miniaturized arrays of points upon a surface. The surface is generally planar. Molecules, including biomolecules, may be attached or synthesized in situ at the points. The attachment points are usually in a column and row format. Other formats may be used. Microarrays are available in different formats and have different surface chemistry characteristics. The different formats and surface characteristics lead to different approaches for attaching or synthesizing molecules. Differences in microarray surface chemistry lead to differences in preparation methods. The attachment points on microarrays are of a micrometer scale, which is generally 1-100 μm.

The term “reactive surface” means a solid surface having chemical functionality that allows a chemical species to “attach” to the reactive surface by physical bonding, and chemical bonding, and combinations thereof. Chemical bonding includes van der Waals forces (dispersion forces and dipole forces), electron donor-acceptor interactions, metallic coordination/complexation, covalent bonding, or combinations thereof. Examples of reactive surfaces include but are not limited to chemical species attached to a surface and having hydroxyl groups, amine groups, or thiol groups. The reactive surface can be porous.

The term “DNA” means deoxyribonucleic acid as a single strand or as a double stranded structure.

The term “RNA” means ribonucleic acid either as a single strand or as a double stranded structure.

The term “nucleotide” means one DNA unit (A, C, G, T) or one RNA unit (A, C, G, U.)

The term “target DNA” means a DNA sequence from a selected gene or genomic sequence of interest. The term gene includes the 3′ untranslated region (UTR) of a gene (RNA). The 3′ UTR is a surrogate for the presence of a gene. Target DNA may be amplified. Amplification may be by polymerase chain reaction (PCR.) A target DNA may be copy DNA (cDNA) made from a RNA sequence. The cDNA may be amplified by PCR.

The term “target RNA” means a RNA sequence from a selected gene or genomic sequence of interest. The term gene includes the 3′ untranslated region (UTR) of a gene. The 3′ UTR is a surrogate for the presence of a gene. Target RNA may have an identifying tag attached thereto.

The term “target” is defined by the terms selected from the group consisting of target DNA and target RNA, and combinations thereof.

The term “tagged target DNA” means a single strand of target DNA. The DNA sequence of the tag is known. The tag may be attached during PCR or attached by some other means. Knowing the sequence of nucleotides at the 3′ end of a DNA sequence is sufficient to constitute a tag. The known sequence may be approximately 10 or more nucleotides. Copy DNA (cDNA) obtained from a RNA sequence is included in the term tagged target.

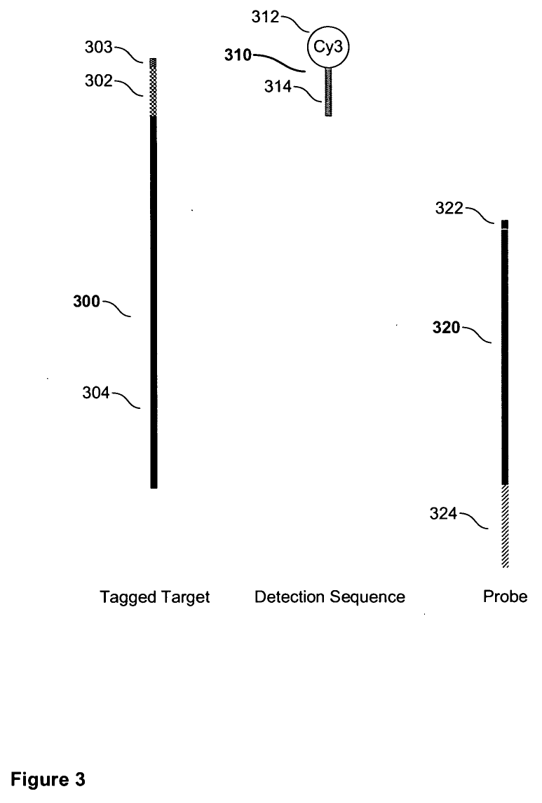

The term “tagged target RNA” means a single strand of target RNA. The RNA sequence of the tag is known. The tag may be inherent in the target such as a polyadenylate near the 3′ end of a RNA strand. Messenger RNA (mRNA) is included in the term tagged target RNA. The length of mRNA may be approximately 50 to 1000 nucleotides in length. Tagged target RNA is used directly in the hybridizing solution rather than first creating cDNA. Referring to FIG. 3, tagged target 300 may be mRNA, which has a polyadenylate that acts as a tag on the three prime end 303 of the tagged target. Detection sequence 310 may comprise a polydeoxythymidylate or a polyuridylate 314 to hybridize to the polyadenylate on the mRNA 300 in addition to a label 312. A label 312 is optional.

The term “tagged target” is defined by the terms selected from the group consisting of tagged target DNA and tagged target RNA, and combinations thereof.

The term “probe DNA” means a single stranded DNA sequence. The sequence of a probe DNA is known. A probe DNA is on a microarray and is attached by in situ synthesis or by spotting. There may be more than one type of probe DNA on a microarray. A probe DNA is generally less than approximately 100 nucleotides. Probe DNA may be optionally attached to a spacer that is attached to a microarray. The spacer may be a nucleotide based spacer, PEG spacer, or another type of spacer.

The term “probe RNA” means a single stranded RNA sequence. The sequence of a probe RNA is known. A probe RNA is on a microarray and is attached by in situ synthesis or by spotting. There may be more than one type of probe RNA on a microarray. A probe RNA is generally less than approximately 100 nucleotides. Probe RNA may be optionally attached to a spacer that is attached to a microarray. The spacer may be a nucleotide based spacer, PEG spacer, or another type of spacer.

The term “probe” is defined by the terms selected from the group consisting of probe DNA, probe RNA, and combinations thereof.

The term “terminal nucleotide DNA” means the nucleotide at the 5′ end of a probe DNA attached to a microarray at its 3′ end.

The term “terminal nucleotide RNA” means the nucleotide at the 5′ end of a probe RNA attached to a microarray at its 3′ end.

The term “terminal nucleotide” is defined by the terms selected from the group consisting of terminal nucleotide DNA and terminal nucleotide RNA, and combinations thereof.

The term “target nucleotide DNA” means the nucleotide on a tagged target DNA that is paired opposite to the terminal nucleotide DNA.

The term “target nucleotide RNA” means the nucleotide on a tagged target RNA that is paired opposite to the terminal nucleotide RNA.

The term “target nucleotide” is defined by the terms selected from the group consisting of target nucleotide DNA and target nucleotide RNA, and combinations thereof.

The term “detection sequence DNA” means a single stranded DNA sequence that is substantially complementary to a sequence on a portion of a tagged target. The detection sequence DNA may have on its 5′ end a label, such as a fluorescent label, that allows the detection sequence DNA to be observed on a microarray when attached to the microarray via ligation to a probe on the microarray.

The term “detection sequence complement DNA” means the sequence on a tagged target DNA that is complementary to a detection sequence. The detection sequence complement DNA is substantially in a 3′ half of the tagged target DNA.

The term “detection sequence RNA” means a single stranded RNA sequence that is substantially complementary to a sequence on a portion of a tagged target. The detection sequence RNA may have on its 5′ end a label, such as a fluorescent label, that allows the detection sequence RNA to be observed on a microarray when attached to the microarray via ligation to a probe on the microarray.

The term “detection sequence complement RNA” means the sequence on a tagged target RNA that is complementary to a detection sequence. The detection sequence complement RNA is substantially in a 3′ half of the tagged target RNA.

The term “detection sequence” is defined by the terms selected from the group consisting of detection sequence DNA, detection sequence RNA, and combinations thereof. If the detection sequence does not have a label, then a subsequent detection step is required to locate microarray locations having ligation of the detection sequence DNA to a probe. A nucleotide having a sequence that is the same as the detection sequence compliment and a label can be used to hybridize to a detection sequence ligated to a probe and remaining after washing.

The term “detection sequence complement” is defined by the terms selected from the group consisting of detection sequence complement DNA and detection sequence complement RNA, and combinations thereof.

The term “hybridized sequence DNA” means the sequence on a tagged target DNA that hybridizes to a probe.

The term “hybridized sequence RNA” means the sequence on a tagged target RNA that hybridizes to a probe.

The term “hybridized sequence” is defined by the terms selected from the group consisting of hybridized sequence DNA and hybridized sequence RNA, and combinations thereof.

The term “hybridized structure DNA” means a double stranded DNA structure. A hybridized structure DNA comprises a tagged target DNA hybridized to a probe DNA and a detection sequence DNA hybridized to the tagged target DNA. After a tagged target DNA hybridizes to a probe DNA and the detection sequence DNA hybridizes to the tagged target DNA, the detection sequence DNA is extended. The detection sequence DNA is ligated to the probe DNA if the terminal nucleotide DNA is complementary to the target nucleotide DNA.

The term “hybridized structure RNA” means a double stranded RNA structure. A hybridized structure RNA comprises a tagged target RNA hybridized to a probe RNA and a detection sequence RNA hybridized to the tagged target RNA. After a tagged target RNA hybridizes to a probe RNA and the detection sequence RNA hybridizes to the tagged target RNA, the detection sequence RNA is extended. The detection sequence RNA is ligated to the probe RNA if the terminal nucleotide RNA is complementary to the target nucleotide RNA.

The term “hybridized structure DNA/RNA” means a double stranded structure comprising hybridized strands of DNA and RNA. A hybridized structure DNA/RNA comprises a tagged target hybridized to a probe and a detection sequence hybridized to the tagged target. After a tagged target hybridizes to a probe and the detection sequence hybridizes to the tagged target, the detection sequence is extended. The detection sequence is ligated to the probe if the terminal nucleotide is complementary to the target nucleotide.

The term “hybridized structure” is defined by the terms selected from the group consisting of hybridized structure DNA, hybridized structure RNA, and hybridized structure DNA/RNA, and combinations thereof.

The term “non-bound material” means material that is not attached to a microarray surface. Non-bound material includes a tagged target hybridized to a probe and a detection sequence that is not ligated to a probe.

The term “match probe” means a single stranded DNA having a specifically designed sequence that is substantially complementary to a hybridized sequence DNA. Match probes are attached to a microarray at known locations. The complementary part of a match probe is the “match sequence.” Match probes generally have no more than approximately four base mismatches with a hybridized sequence DNA. The terminal nucleotide DNA of a match probe must be complementary to the target nucleotide DNA of a tagged target DNA when hybridized in order to allow ligation. Match probe is included in the term “probe DNA.”

The term “mismatch probe” means single stranded DNA having a specifically designed sequence that is substantially complementary to the hybridized sequence DNA. Mismatch probes are attached to a microarray at known locations. The complementary part of a mismatch probe is the “mismatch sequence.” Mismatch probes generally have no more than approximately four base mismatches when hybridized to a tagged target DNA. The terminal nucleotide DNA of a mismatch probe must not be complementary to the target nucleotide DNA of a tagged target DNA when hybridized in order to prevent ligation. The mismatch probes and the match probes have the same base sequence except for the terminal nucleotide DNA. Mismatch probe is included in the term “probe DNA.”

The term “match structure” means a double stranded DNA structure, wherein a match probe bound to a microarray is hybridized to a tagged target DNA and a detection sequence DNA is hybridized to the tagged target DNA. “Match structure” is included in the term “hybridized structure DNA.”

The term “mismatch structure” means a double stranded DNA structure, wherein a mismatch probe bound to a microarray is hybridized to a tagged target DNA and a detection sequence DNA is hybridized to the tagged target DNA. “Mismatch structure” is included in the term “hybridized structure DNA.”

The term “set probes” means approximately three to four probes that are identical except that the terminal nucleotide is one of the four nucleotides, i.e., A, C, G, or T. Thus, there are approximately three to four probes with each probe nearly identical except the terminal nucleotide DNA. “Set probes” is included in the term “probe DNA.”

The term “set structures” means a double stranded DNA structure. Each set structure comprises a tagged target DNA hybridized to a one of the set probes and a detection sequence DNA hybridized to the detection sequence complement DNA of the tagged target DNA. After a tagged target DNA hybridizes to a set probe and the detection sequence DNA hybridizes to the tagged target DNA, the detection sequence is extended. The detection sequence DNA ligates to one of the set probes if the terminal nucleotide DNA is complementary to the target nucleotide DNA. “Set structures” is included in the term “hybridized structure DNA.”

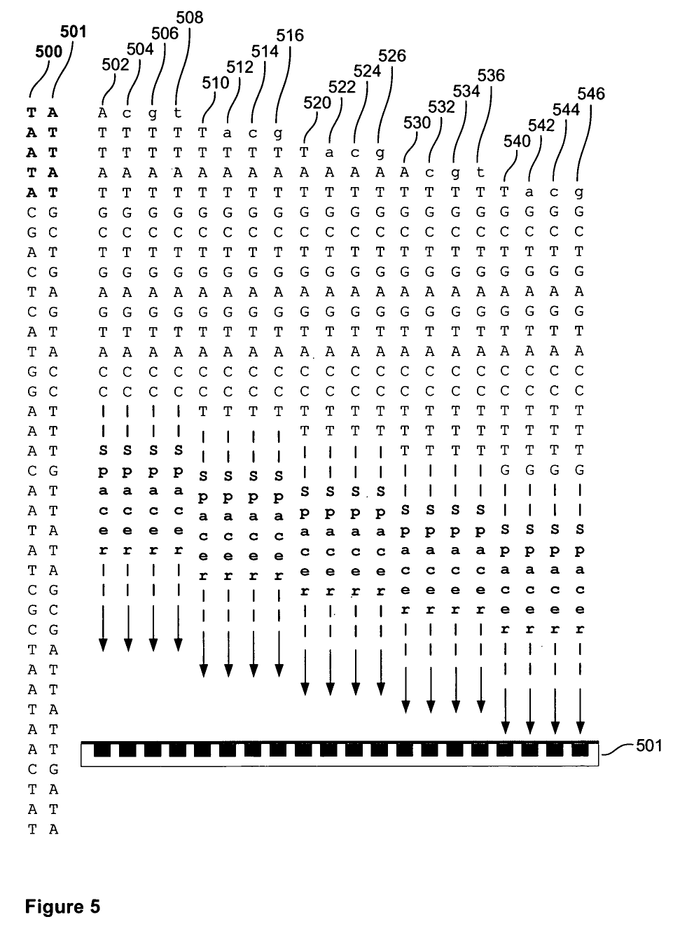

The term “consecutive sequence probe” are probes that span the entire DNA sequence of interest by stepping one or more base units at a time along the length of the sequence to represent each base of interest in the sequence as a terminal base on the probes. FIG. 5 is a schematic of the sequences selected for consecutive sequence probes of an example sequence. Referring to FIG. 5, sequential complementary probes 502, 510, 520, 530, 540 are obtained by stepping along the sequence of interest 500 one base at a time. The probes are based on the complementary sequence 501. Other step sizes may be used, such as 5 bases at a time. Each sequential complementary probe has accompanying identical probes except for the terminal sequence. For example, probe 502 has probes 504, 506, 508 accompanying probe 502. Probes are the complement of the tagged target. At each base along the sequence of interest, there are four probes that are identical except for the terminal nucleotide is A, C, G, or T to allow determination of the base on the target at each location along the sequence of interest. Less than four probes may be used for each base. Consecutive sequence probe is included in the term “probe DNA.”

The term “consecutive sequence structures” includes hybridized structures formed between the consecutive sequence probes, a tagged target, and a detection sequence. The detection sequence is extended and ligated to the probe in a consecutive sequence structure when the terminal nucleotide is the complement of the target nucleotide. Consecutive sequence structures are included in the term “hybridized structure DNA.”

The term “gene expression probes” means probes having sequences designed to be complementary to a sequence within a gene of interest or a sequence that is a surrogate for the gene of interest such as the 3′ UTR. There may be as many gene expression probes as there are genes of interest in a particular genome or sequence. Gene expression probe is included in the term “probe DNA.”

The term “gene expression structures” means a double stranded DNA structure. Each gene expression structure comprises a tagged target hybridized to a one of the gene expression probes and a detection sequence hybridized to the detection sequence complement of the tagged target. After a tagged target hybridizes to a gene expression probe and the detection sequence hybridizes to the tagged target, the detection sequence is extended and then ligated to a gene expression probe if the terminal nucleotide is the complementary base to its pair on the hybridized sequence. The degree of ligation is a measure of the degree of gene expression in the tagged target.

The term “dNTP” means a solution of deoxynucleotide triphosphate mix of deoxyadenine triphosphate (dATP), deoxycytosine triphosphate (dCTP), deoxyguanine triphosphate (dGTP), and deoxythymine triphosphate (dTTP).

The term “rNTP” means a solution of nucleotide triphosphate mix of adenine triphosphate (rATP), cytosine triphosphate (rCTP), guanine triphosphate (rGTP) and uracil triphosphate (rUTP).

The term “triphosphate mix” includes dNTP and rNTP.

The present invention provides a SNP, sequencing, and gene expression assay method on a microarray. A microarray is provided having a plurality of probes on the microarray. A plurality of hybridized structures is formed on the microarray. Each hybridized structure is formed by contacting the microarray under a hybridizing condition to a hybridizing solution comprising a plurality of tagged targets and a plurality of detection sequences. Each hybridized structure comprises one tagged target hybridized to one probe and to one detection sequence. Each hybridized structure is extended using an extension-ligation solution and an extension-ligation condition. Each hybridized structure having a terminal nucleotide that is complementary to a target nucleotide is ligated using the extension-ligation solution and the extension-ligation condition. Non-bound material is removed by washing the microarray using a wash solution and a wash method. The target nucleotide and a hybridized sequence from the hybridized structures having ligation are identified.

In a preferred embodiment, the detection sequence has a label. The label provides identification of microarray spots where a detection sequence is ligated to a probe. Preferably, the label is a fluorescent label. Locations having the label are identified by fluorescence imaging of the microarray. Other labels may be used and such a label may be detectable by spectroscopic, photochemical, biochemical, immunochemical, electrical, optical, laser, or chemical means.

In an alternative embodiment, the detection sequence does not have a label. Identification comprises adding a labeled material to the microarray after washing. In one embodiment, a solution having an oligonucleotide having a sequence substantially identical to a detection sequence complement and having a label, such as a fluorescent label, is contacted to the microarray. The sequence is hybridized to the detection sequences remaining after washing. The remaining detection sequences are those ligated to a probe. A hybridization solution is used to hybridize the oligonucleotide to the detection sequences. The concentration of the oligonucleotide is 1 micromolar. Any standard hybridization buffer is a suitable buffer for hybridization. For example, 3× SSPE (phosphate buffered saline with EDTA) or 3× SSC (citrate buffered saline) are suitable buffers. The solution having the oligonucleotide is added to the microarray hybridization chamber and incubated at approximately 30° C. to 55° C. for approximately 0.5 to 1.5 hours. Preferably, the incubation is approximately 45° C. for approximately 1 hour. The microarray locations having hybridization of the oligonucleotide having the label are identified by fluorescence imaging of the microarray.

In one embodiment of the present invention, a target is selected from a genome of interest. The target sequence is amplified using PCR and suitable primers or some other suitable method. One of the primers contains a common tag for re-amplification and detection. The common tag, which is added during amplification, provides the template for primer extension and an anti-sense sequence for labeled primer hybridization. After amplification, the combined single-stranded PCR product and labeled common primer are hybridized on a microarray. A single step extension and ligation reaction is performed, which can be accomplished using a DNA ligase such as E. coli DNA ligase and a polymerase such as Taq Stoffel fragment or reverse transcriptase. The enzymes for this combined reaction must have narrowly defined characteristics. First, the polymerase must not have strand displacement or exonuclease activity. Second, the ligase must be able to discriminate a mismatch. A final wash step removes un-ligated material and allows determination of a SNP, sequence, or gene expression. In the preferred embodiment, room temperature 0.1 molar sodium hydroxide is used for washing the microarray to remove unbound material.

In another embodiment of the present invention, an RNA target sequence is selected from a target sequence instead of a DNA target sequence. Prior to PCR as used in the method for a DNA target sequence, a cDNA is made from the RNA target sequence using reverse transcriptase. The target sequence and two primers are amplified using PCR or some other suitable method. One of the primers contains a common tag for re-amplification and detection. The common tag, which is added during amplification, provides the template for primer extension and an anti-sense sequence for labeled primer hybridization. After amplification, the combined single-stranded PCR product and labeled common primer are hybridized on a microarray. A single step extension and ligation reaction is performed, which can be accomplished using a DNA ligase such as E. coli DNA ligase and a polymerase such as Taq Stoffel fragment or reverse transcriptase. The enzymes for this combined reaction must have narrowly defined characteristics. First, the polymerase must not have strand displacement or exonuclease activity. Second, the ligase must be able to discriminate a mismatch. A final 65° C. water wash step removes un-ligated label and allows determination of a SNP, sequence, or gene expression. In the preferred embodiment, room temperature 0.1 molar sodium hydroxide is used for washing the microarray to remove unbound material.

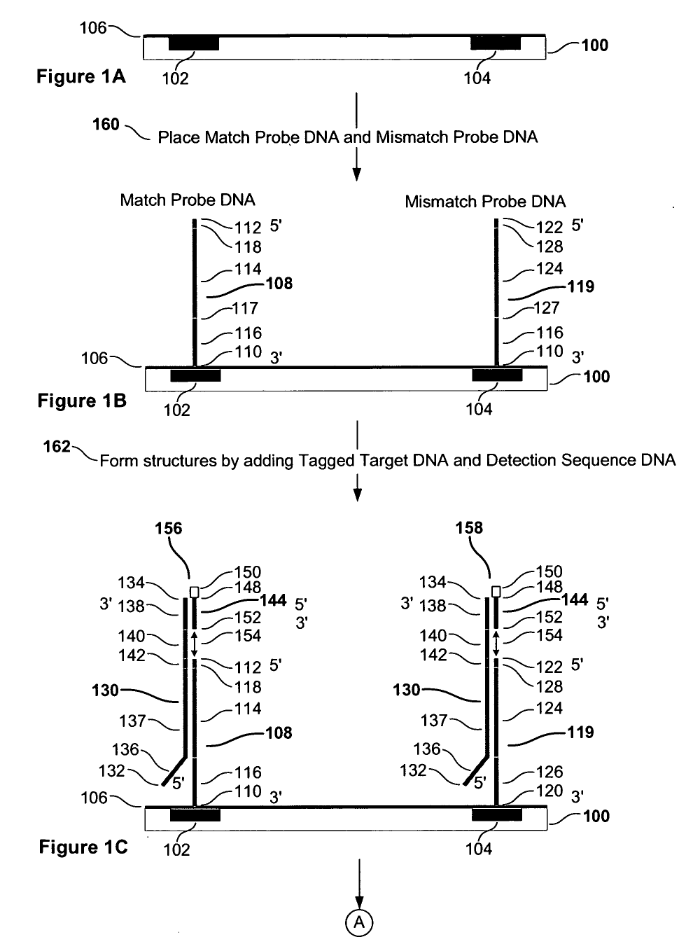

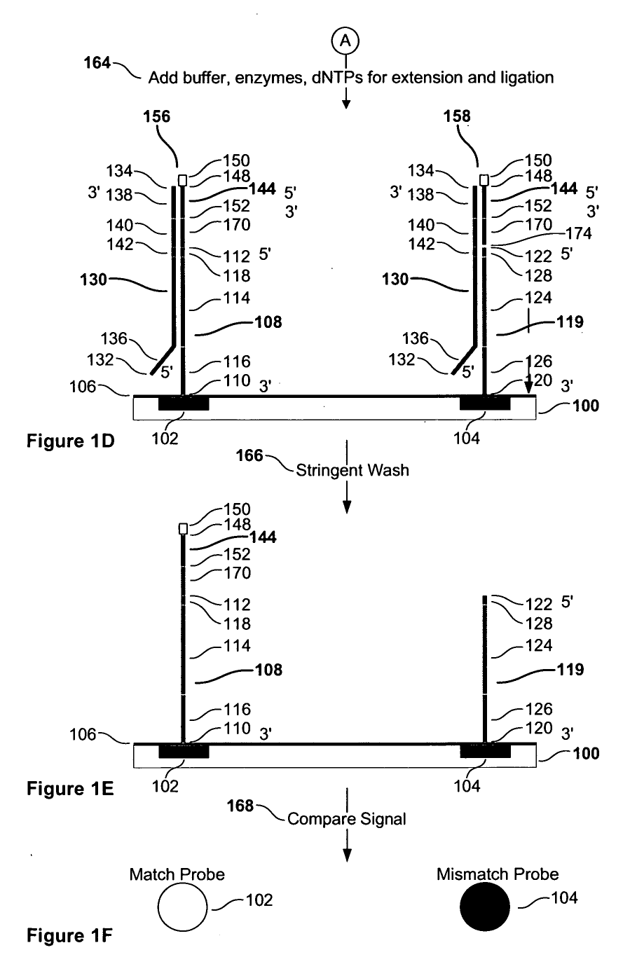

An embodiment of the present invention is shown in FIGS. 1A through 1F as a schematic of a portion of a microarray after a sequence of steps. FIG. 1A is a schematic of a cross section of a microarray 100 having a plurality of locations 102, 104 and having a reactive surface 106 for attachment of probes. The reactive surface 106 may cover the entire array or only the locations 102, 104.

Referring to FIG. 1B, a plurality of match probes 108 is placed 160 on at least one match location 102 on the microarray 100, and a plurality of mismatch probes 119 is placed 160 on at least one mismatch location 104 on the microarray 100. The match probes 108 have a match 3′ end 117, a match 5′ end 112, and a match terminal nucleotide 118 on the match 5′ end 112. The match terminal nucleotide 118 is the last nucleotide on the match 5′ end 112. The mismatch probes 119 have a mismatch 3′ end 127, a mismatch 5′ end 122, and a mismatch terminal nucleotide 128 on the mismatch 5′ end 122. The mismatch terminal nucleotide 128 is the last nucleotide on the mismatch 5′ end 122. The match 3′ end 117 and the mismatch 3′ end 127 are attached to an optional spacer 116 having attachment end 110 attached to the microarray 100 at locations 102, 104 respectively to the reactive surface 106.

The match probes 108 and the mismatch probes 119 have the same nucleotide sequence 114, 124 except the match terminal nucleotide 118 is different from the mismatch terminal nucleotide 128. The match terminal nucleotide 118 may be wild type or mutated type. If the match terminal nucleotide is wild type, then the mismatch terminal nucleotide is mutated type. If the match terminal nucleotide is mutated type, then the mismatch terminal nucleotide is wild type.

Preferably the microarray is an electrode microarray. Preferably the match probes 108 and the mismatch probes 119 are synthesized in situ on an electrode microarray. Preferably in situ synthesis of the match probes 108 and the mismatch probes 119 is performed using standard phosphoramidite chemistry and electrochemical deblocking. Preferably, the probes are approximately 5 to approximately 100 nucleotides in length. More preferably, the probes are approximately 10 to approximately 50 nucleotides in length. Most preferably, the probes are approximately 15 to approximately 30 nucleotides in length.

Preferably the match and mismatch probes 108, 119 are attached to a spacer 116 having approximately 1 to 50 nucleotides. More preferably, the spacer 116 has approximately 3 to 25 nucleotides. Most preferably, the spacer 116 has approximately 5 to 15 nucleotides. However, a spacer is not required to practice the present invention. Probes attached without a spacer fall within the scope of the present invention. Without being bound by theory, a spacer likely provides better access of a tagged target to a probe for hybridization of the tagged target to the probe. A spacer comprised of a chemical species other than DNA falls within the scope of the present invention. Examples of such suitable spacers include but are not limited to modified DNA, RNA, modified RNA, peptides, polyethylene glycols (PEG), and PEG derivatives, and combinations thereof. Any chemical species that is suitable as a spacer for attachment of DNA to a microarray for the practice of the present invention falls within the scope of the present invention.

Referring to FIG. 1C, after placement of match probes 108 and mismatch probes 119 on the microarray 100 with the optional spacer 116, a plurality of match structures 156 are formed on at least one match location 102 and a plurality of mismatch structures 158 are formed on the at least one mismatch location 104 by addition of tagged target DNA 130 and detection sequence DNA 144 as shown in FIG. 1C. The plurality of match structures 156 and the plurality of mismatch structures 158 are formed by contacting the microarray 100 under a hybridizing condition to a hybridizing solution comprising a plurality of tagged target DNA 130 and a plurality of detection sequence DNA 144.

The hybridizing condition depends on solution parameters termed stringency. Stringency includes temperature, salt content and valence, and detergent content as well as other parameters. Controlling stringency allows control of the extent of hybridization. For example, at a lower temperature, single stranded oligonucleotide has a higher probability of bonding to another single strand oligonucleotide, whereas at higher temperature, there is less probability of bonding given all else being equal. In the preferred embodiment, the stringency is selected such that hybridization occurs between detection sequences and tagged targets and between tagged targets and probes.

Preferably, there are at most approximately 3 to 4 mismatches between hybridizing strands. More preferably, there is at most one mismatch between a tagged target DNA and a mismatch probe, where the mismatch is located at the terminal nucleotide DNA of the mismatch probe. More preferably, there are no mismatches between the tagged target DNA and the match probe. Preferably, the hybridizing solution comprises a plurality of tagged target DNA and a plurality of detection sequence DNA in a buffer solution. Preferably, the buffer solution is 1×T4 ligase buffer.

Each of the plurality of tagged target DNA 130 has a target 3′ end 134, a target 5′ end 132, a match sequence 137 substantially complementary to the match probes 114, and a detection sequence complement DNA 138 substantially complementary to the detection sequence DNA 144. The match sequence 137 has a target nucleotide DNA 142 where a single nucleotide polymorphism will be located when the tagged target DNA has the single nucleotide polymorphism. The tagged target DNA 130 has the detection sequence complement DNA 138 towards the tagged target DNA 3′ end 134 and the match sequence 137 towards the tagged target DNA 5′ end 132. The detection sequence DNA 144 has a 5′ detection end 148 and a 3′ extension end 152. The 5′ detection end has a tag or label 150. Preferably, the tag 150 is a fluorescent (Cy3) tag. However, any fluorescent tag is suitable. Other tags may be used. Preferably, the detection sequence complement DNA 138 is a T7-based oligonucleotide [SEQ ID NO:5 or SEQ ID NO:6].

Each of the plurality of match structures 156 comprises one of the plurality of tagged target DNA 130 hybridized to one of the plurality of match probes 108 and one of the plurality of detection sequence DNA 144 hybridized to one of the plurality of tagged target DNA 130. Hybridization occurs such that the match terminal nucleotide 118 is paired to and complementary to the target nucleotide DNA 142 of the tagged target DNA 130. Each of the plurality of match structures 156 has the 3′ extension end 152 of the detection sequence DNA 144 facing the match 5′ end 112 of the match probes 108.

Each of the plurality of mismatch structures 158 comprises one of the plurality of tagged target DNA 130 hybridized to one of the plurality of mismatch probes 119 and one of the plurality of detection sequence DNA 144 hybridized to one of the plurality of tagged target DNA 130. Hybridization occurs such that the mismatch terminal nucleotide 128 is paired to but not complementary to the target nucleotide 142 of the tagged target DNA 130. Each of the plurality of mismatch structures 158 has the 3′ extension end 152 of the detection sequences 144 facing the match 5′ end 122 of the mismatch probes 119.

Each match structure 156 and each mismatch structure 158 has a single stranded sequence 140 that is not hybridized. Each match structure 156 and each mismatch structure 158 may have a non-hybridized tail 136. Whether the non-hybridized tail 136 exists depends upon which part of the tagged target DNA 130 is of interest for a particular experiment to determine whether a SNP is present in the tagged target DNA 130. If a sequence of the tagged target DNA 130 of interest includes the target 5′ end 132, then the match probes 108 will be complementary to the sequence that includes the target 5′ end 132 such that the non-hybridized tail 136 will not be present. The length of the non-hybridized tail 136 depends upon which sequence of the tagged target DNA 130 is hybridized to the probes.

Referring to FIG. 1D, after formation of match structures 156 and mismatch structures 158, the microarray 100 is contacted to an extension-ligation solution under an extension-ligation condition. Preferably, the extension-ligation solution comprises a mixture of polymerase, ligase, dNTPs (deoxynucleotide triphosphate mix of dATP, dCTP, dGTP, and dTTP,) and buffer. Preferably, the extension-ligation condition comprises a temperature of approximately 37° C. for one hour. Under such conditions, each match structure 3′ extension end 152 is extended towards the match structure 5′ prime end 112 of the plurality of match structures 156 thus forming an extension sequence 170 hybridized to the single nucleotide sequence 140 of each match structure 156. The match terminal nucleotide 118 ligates to the extension sequence 170 of the plurality of match structures 156. Additionally, under such conditions, each mismatch structure 3′ extension end 152 is extended towards the mismatch structure 5′ end 122 of the plurality of mismatch structures 158 thus forming an extension sequence 170 hybridized to the single nucleotide sequence 140 of each mismatch structure 158. The mismatch terminal nucleotide 128 prevents ligation of the extension sequence 170 to mismatch terminal nucleotide 128 of the plurality of mismatch structures 158 because the target nucleotide 142 is not complementary to the mismatch terminal nucleotide 128.

Referring to FIG. 1E, after ligation in the match structures 156 and no ligation in the mismatch structures 156, the non-bound material is removed by washing the microarray 100 using a wash solution and a wash method. The wash solution is selected from the group consisting of buffer solution and base solution. Preferably, the buffer is a Tris buffer or a phosphate buffer, although other buffers are suitable. Preferably, the phosphate buffer is a phosphate buffered saline (PBS) having a pH of approximately 7 to 7.5. Preferably, the Tris buffer is a Tris-HCl having a pH of approximately 7 to 7.5. Preferably, the PBS buffer is 0.05×PBS buffer. Preferably, the base solution is an aqueous sodium hydroxide solution. Preferably, the concentration of base is approximately 0.01 to 5 molar. More preferably, the base concentration is approximately 0.05 to 1 molar. Most preferably, the base concentration is approximately 0.1 molar sodium hydroxide. The wash method comprises exposing the microarray to the wash solution at a temperature of approximately room temperature to approximately seventy degrees centigrade. Preferably, the wash method comprises exposing the microarray to the wash solution having base at approximately room temperature until sufficient non-bound material is removed to allow accurate reading of the microarray. Generally, washing approximately three times is sufficient. The detection sequences 144 of the plurality of mismatch structures 158 is substantially removed, and the detection sequences 144 ligated to the match probes of the plurality of match structures is substantially not removed. The tagged targets 130 of the match structures 156 and the mismatch structures 158 may be removed. However, the present invention does not require removal of the tagged target DNA 130 from the match structures 156 and mismatch structures 158.

Referring to FIG. 1F, after washing, the microarray is examined to determine the microarray locations where the match structures are located. The location of the match structures is determined by the tag 150 on the detection sequences 144 because the detection sequence DNA 144 is ligated to the match probes 108. Since the match locations have the tag 150 and the mismatch locations do not have the tag 150, the match locations have a high reading while the mismatch locations have a low reading. The high reading identifies the match terminal nucleotide 118, which is used to identify the target nucleotide 142 because the match terminal nucleotide 118 and the target nucleotide DNA 142 are complementary to each other. The target nucleotide DNA 142 is identified by the match terminal nucleotide 118 because the match terminal nucleotide is known. The target nucleotide DNA 142 can be compared to a known single nucleotide polymorphism to determine whether the plurality of tagged targets 130 has the known single nucleotide polymorphism. Alternatively, the target nucleotide is used to identify a base in a sequence. Alternatively, the target nucleotide is used to identify gene expression.

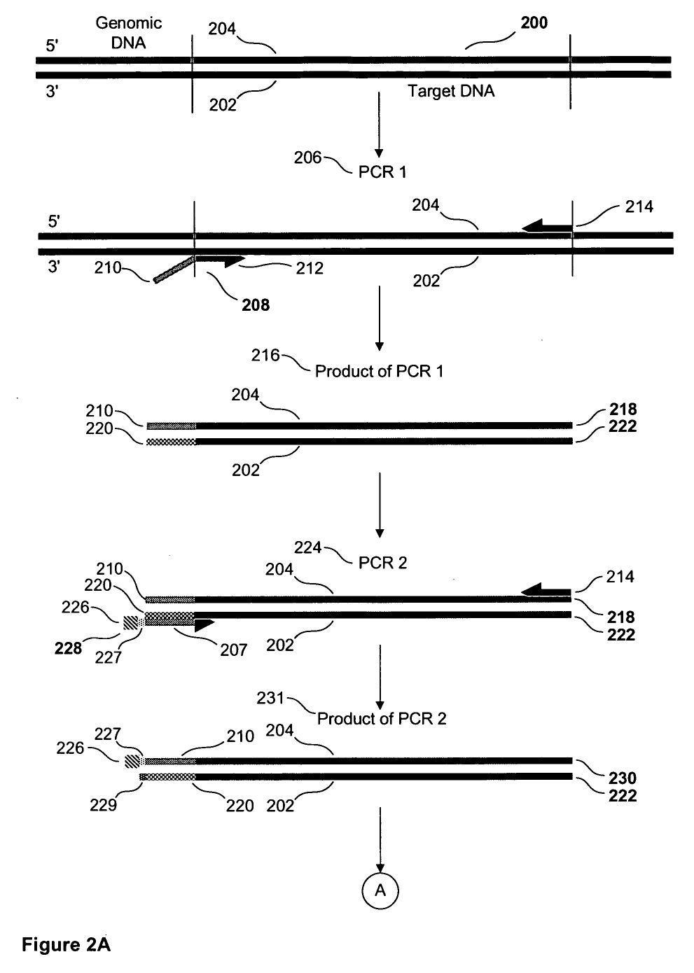

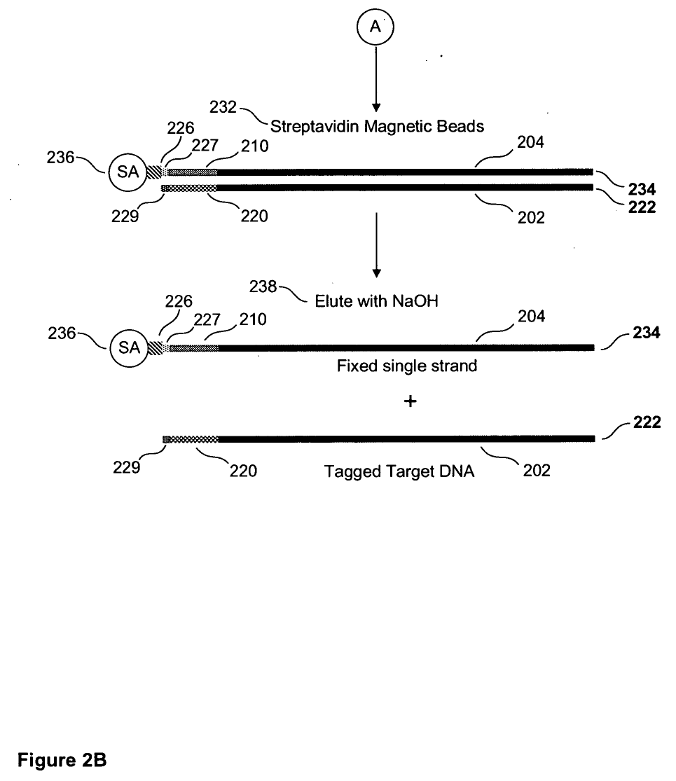

In a preferred embodiment, the tagged target DNA 130 (FIG. 1C) is obtained by polymerase chain reaction (PCR) amplification of a portion of genomic DNA. The genomic DNA has at least one SNP of interest is located if the SNP is present in the genomic DNA. Alternatively, the genomic DNA is a sequence to be identified. Alternatively, the genomic DNA is a gene of interest to determine the degree of gene expression. Referring to FIGS. 2A and 2B, a target genomic DNA 200 is selected. Target DNA 202 having complement 204 is selected. The target DNA 202 and the complement 204 are amplified by a first stage PCR amplification 206 using a tagged specific forward primer 208 and a specific reverse primer 214. The tagged specific forward primer 208 has a tag 210 and a forward primer 212. The product of the first stage PCR 216 has the tagged target DNA 222 having a tag complement 220 and the target 202. In addition, the product 216 has the complement of the tagged target DNA 218 having a tag 210 and the complement of the target 204.

A second stage PCR 224 is used to introduce a biotin tag 226. A specific reverse primer 214 and a biotin-tagged specific forward primer 228 are used in the second stage. The forward primer has a biotin tag 226 attached to a primer 207, which is the same DNA sequence as tag 210 but has an optional DNA extension 227. The product of the second stage PCR 228 has the tagged target DNA 222 having a complement tag 220, optional complement of the optional DNA extension 229 and the target 202. In addition, the product 228 has the complement of the tagged target DNA 230 having a tag 210 having an optional DNA extension 227, the complement of the target 204, and a biotin tag 226 attached to the tag 210. In the next step, magnetic beads having streptavidin 232 are added to attach the biotin 226 to the beads 236, thus anchoring the amplified DNA double strands 222, 234. The tagged target DNA 222 is recovered by eluting with NaOH 238. The tagged target DNA 222 is eluted, and the complement remains attached to the magnetic beads 236 via the biotin 226.

Referring to FIG. 3, a microarray having attached probes 320, an optional spacer 324 and terminal nucleotide 322 is exposed to a solution containing the tagged target 300 having target 304 and complement tag 302 combined with detection sequence 310 comprising a fluorescent dye (Cy3) 312 and tag 314.

Referring to FIG. 1B, the match terminal nucleotide 118 may be complementary to wild type or a mutated type. If the match terminal nucleotide 118 is complementary to wild type, then the mismatch terminal nucleotide 128 is complementary to a mutated type, and since the target nucleotide 142 has a match to the wild type, then the target does not have the mutation. In contrast, if the match terminal 118 is mutated type, then the mismatch terminal nucleotide 128 is wild type, and since the target has a match to the mutated type, then the target has the mutation.

In another embodiment of the present invention, the match probes 108 and the mismatch probes 119 are pre-synthesized and then placed onto the microarray. Suitable placement methods include but are not limited to (1) spotting a solution on a prepared flat surface using spotting robots and (2) electric field attraction/repulsion deposition. Preferably, the probes are synthesized in situ on an electrode microarray.

In another embodiment of the present invention, the match probes 108 and the mismatch probes 119 are synthesized in situ thereon using a method that does not require use of an electrode microarray. Such suitable in situ synthesis methods include but are not limited to (1) in situ synthesis by printing reagents via ink jet or other printing technology and using standard phosphoramidite chemistry, (2) maskless photo-generated acid (PGA) controlled in situ synthesis and using standard phosphoramidite chemistry, (3) mask-directed in situ parallel synthesis using photo-cleavage of photolabile protecting groups (PLPG), and (4) maskless in situ parallel synthesis using PLPG and digital photolithography.

In another embodiment of the present invention, only a plurality of match probes 108 is placed on a microarray 100 on at least one match location 102. The terminal nucleotide 118 is used to identify the target complement 142. If the target complement 142 is mutated, then the target has the SNP of interest. If the target complement 142 is wild type, then the target does not have the SNP of interest.

In another embodiment of the present invention, only mismatch probes 119 are placed on a microarray 100 on at least one mismatch location 104. The terminal nucleotide 128 is used to identify the target nucleotide 142 by a process of elimination. Since detection sequence DNA 310 will not ligate when target nucleotide 142 and terminal nucleotide 128 are not complementary, target nucleotide 142 can be identified by the terminal nucleotides 128 that are not complementary to target nucleotide 142. For example, if target nucleotide 142 is an A, then terminal nucleotides 128 A, C, and G will prevent ligation so that target nucleotide 117 can be identified as A by a process of elimination.

Preferably, target and probes have less than five internal mismatches, and target and probes have less than five internal mismatches. Preferably, the extension-ligation solution comprises water, E. coli ligase buffer, dNTP, Taq polymerase Stoffel fragment, and E. coli ligase. More preferably, the extension-ligation solution comprises approximately 155 microliters of water, approximately eighteen microliters of ten times concentrated E. coli ligase buffer, approximately three microliters of ten millimolar dNTP, approximately two microliters of Taq polymerase Stoffel fragment, and approximately two microliters of E. coli ligase. Preferably, the extension-ligation condition comprises incubation of the microarray exposed to the extension-ligation solution at approximately thirty-seven degrees centigrade for approximately one hour.

The wash solution is selected from the group consisting of buffer solution and base solution. Preferably, the buffer is a Tris buffer or a phosphate buffer, although other buffers are suitable. Preferably, the phosphate buffer is a phosphate buffered saline (PBS) having a pH of approximately 7 to 7.5. Preferably, the Tris buffer is a Tris-HCl having a pH of approximately 7 to 7.5. Preferably, the PBS buffer is 0.05×PBS buffer. Preferably, the base solution is an aqueous sodium hydroxide solution. Preferably, the concentration of base is approximately 0.01 to 5 molar. More preferably, the base concentration is approximately 0.05 to 1 molar. Most preferably, the base concentration is approximately 0.1 molar sodium hydroxide. The wash method comprises exposing the microarray to the wash solution at a temperature of approximately room temperature to approximately seventy degrees centigrade. Preferably, the wash method comprises exposing the microarray to the wash solution having base at approximately room temperature until sufficient non-bound material is removed to allow accurate reading of the microarray. Generally, washing approximately three times is sufficient.

In another embodiment of the invention, referring to FIG. 1A, additional microarray locations may be used in combination with locations 102, 104. For example, if wild type is an A and mutated type is a C, then locations 102, 104 can be used having a T and a G as terminal nucleotides on the probes. If a mutation can be a C or a G, then an additional location can be used having a probe having a C terminal nucleotide. If a mutation can be a C, G, or T, then two additional locations can be used where the additional locations have C and A as terminal nucleotides on the probes. Thus, up to four locations having probes with different terminal nucleotides may be used to determine the target nucleotide of the tagged targets.

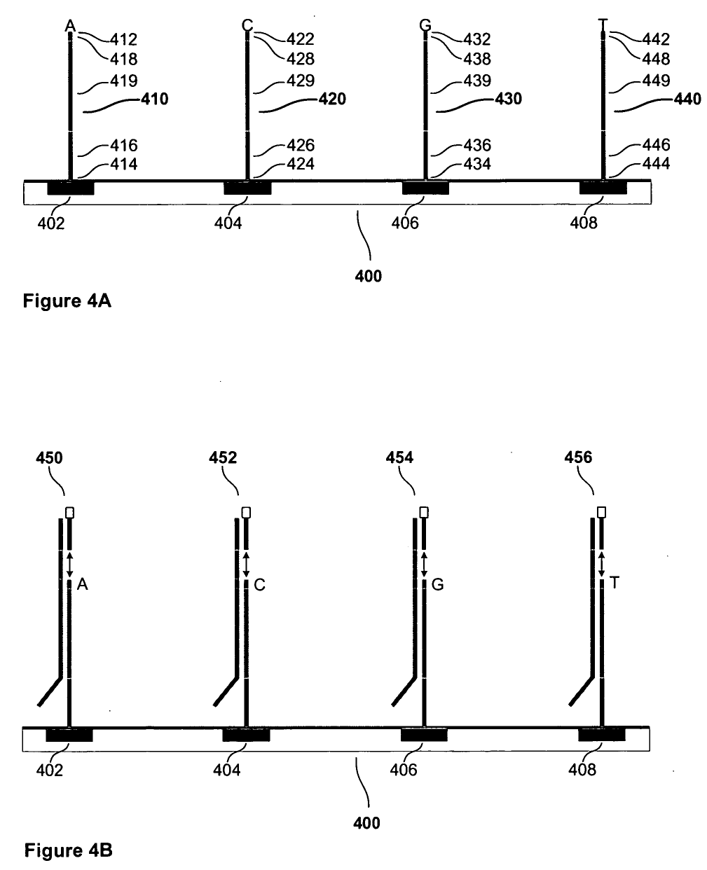

Referring to FIG. 4A, in one embodiment of the present invention, the microarray-based single nucleotide polymorphism, sequencing, and gene expression assay method comprises providing a microarray having four types of probe DNA and using tagged target DNA and detection sequence DNA. The probe DNA comprises a plurality of A-probes 410 on an A-location 402 on the microarray 400, a plurality of C-probes 420 on a C-location 404 on the microarray 400, a plurality of G-probes 430 on a G-location 406 on the microarray 400, and a plurality of T-probes 440 on a T-location 408 on the microarray 400. The A-probes 410 have an A-3′ end 414, an A-5′ end 412, and a deoxyadenylate 418 on the A-5′ end 412. The C-probes 420 have a C-3′ end 424, a C-5′ end 422, and a deoxycytidylate 428 on the C-5′ end 422. The G-probes 430 have a G-3′ end 434, a G-5′ end 432, and a deoxyguanylate 438 on the G-5′ end 432. The T-probes 440 have a T-3′ end 444, a T-5′ end 442, and a deoxythymidylate 448 on the T-5′ end 442. The A-3′ end 414, the C-3′ end 424, the G-3′ end 434, the T-3′ end 444 are attached to the microarray. The A-probes 410, the C-probes 420, the G-probes 430, and the T-probes 440 are approximately identical except for the deoxyadenylate 418 on the A-5′ end 412, the deoxycytidylate 428 on the C-5′ end 422, the deoxyguanylate 438 on the G-5′ end 432, and the deoxythymidylate 448 on the T-5′ end 442. Each probe is shown having an optional spacer 416, 426, 436, 446.

Referring to FIGS. 1C, 3, 4A, and 4B, each probe 410, 420, 430, 440 has a DNA sequence 419, 429, 439, 449 having less than 5 base pair mismatches with the tagged target DNA 300. A plurality of A-structures 450 on the at least one A-location 402, a plurality of C-structures 452 on the at least one C-location 404, a plurality of G-structures 454 on the at least one G-location 406, a plurality of T-structures 456 on the at least one T-location 408 are placed on the microarray 400 by in situ synthesis or by spotting. The A-structures 450, C-structures 452, G-structures 454, and T-structures 456 are formed by contacting the microarray 400 under a hybridizing condition to a hybridizing solution comprising a plurality of tagged target DNA 300 and a plurality of detection sequence DNA 310. Each of the plurality of tagged target DNA 130, 300 has a target 3′ end 134, a target 5′ end 132, a detection sequence complement DNA 138 substantially complementary to the detection sequence DNA 310, 144, and a target sequence DNA 137, 304 substantially complementary to the A-probes 419, the C-probes 429, the G-probes 439, and the T-probes 449. The target sequence 137, 304 has a target nucleotide 142 corresponding to a location in a target genome. The detection sequence complement DNA 138 is on the target 3′ end 134 and the match sequence 137 is on the target 5′ end 132. The detection sequence 144 has a 5′ detection end 148 and a 3′ extension end 152.

Each A-structure comprises one tagged target DNA hybridized to one A-probe and hybridized to one detection sequence DNA. The deoxyadenylate on the A-5′ end is base-paired to the target nucleotide. Each of the plurality of A-structures has the 3′ extension end of the detection sequence DNA facing the A-5′ end of the A-probes providing a A-structure 3′ extension end and a A-structure 5′ end (FIG. 4B, 450.)

Each C-structure comprises one tagged target DNA hybridized to one C-probe and hybridized to one detection sequence DNA. The deoxycytidylate on the C-5′ end is base-paired to the target nucleotide. Each of the plurality of C-structures has the 3′ extension end of the detection sequence DNA facing the C-5′ end of the C-probes providing a C-structure 3′ extension end and a C-structure 5′ end (FIG. 4B, 452.)

Each G-structure comprises one tagged target DNA hybridized to one G-probe and hybridized to one detection sequence DNA. The deoxyguanylate on the G-5′ end is base-paired to the target nucleotide. Each of the plurality of G-structures has the 3′ extension end of the detection sequence DNA facing the G-5′ end of the G-probes providing a G-structure 3′ extension end and a G-structure 5′ end (FIG. 4B, 454.)

Each T-structure comprises one tagged target DNA hybridized to one T-probe and hybridized to one detection sequence DNA. The deoxythymidylate on the T-5′ end is base-paired to the target nucleotide. Each of the plurality of T-structures has the 3′ extension end of the detection sequence DNA facing the T-5′ end of the T-probes providing a T-structure 3′ extension end and a T-structure 5′ end (FIG. 4B, 456.)

The microarray is contacted to an extension-ligation solution under an extension-ligation condition resulting in each A-structure 3′ extension end extending towards the A-structure 5′ end of the plurality of A-structures, each C-structure 3′ extension end extending towards the C-structure 5′ end of the plurality of C-structures, each G-structure 3′ extension end extending towards the G-structure 5′ end of the plurality of G-structures, and each T-structure 3′ extension end extending towards the T-structure 5′ end of the plurality of T-structures. After extension, A-extended detection sequences are formed adjacent to the A-structure 5′ end; C-extended detection sequences are formed adjacent to the C-structure 5′ end; G-extended detection sequences are formed adjacent to the G-structure 5′ end; and T-extended detection sequences are formed adjacent to the T-structure 5′ end (FIG. 1D, 170).

Each A-structure having a terminal nucleotide DNA complementary to the target nucleotide DNA has ligation of the A-extended detection sequence DNA to the A-structure 5′ end. Each C-structure having a terminal nucleotide DNA complementary to the target nucleotide DNA has ligation of the C-extended detection sequence DNA to the C-structure 5′ end. Each G-structure having a terminal nucleotide DNA complementary to the target nucleotide DNA has ligation of the G-extended detection sequence DNA to the G-structure 5′ end. Each T-structure having a terminal nucleotide DNA complementary to the target nucleotide DNA has ligation of the T-extended detection sequence DNA to the T-structure 5′ end.

Non-bound material is removed by washing the microarray using a wash solution and a wash method. The A-extended detection sequence DNA, the C-extended detection sequence DNA, the G-extended detection sequence DNA, and the T-extended detection sequence DNA not ligated are removed. The A-extended detection sequence DNA, the C-extended detection sequence DNA, the G-extended detection sequence DNA, and the T-extended detection sequence DNA ligated are not removed. One of the A-location, the C-location, the G-location, and the T-location have a high reading, and three of the A-location, the C-location, the G-location, and the T-location have a low reading. The high reading location corresponds to a location on the microarray having the target nucleotide DNA having a complementary terminal nucleotide DNA. The low reading corresponds to locations on the microarray having the target nucleotide DNA having a non-complementary terminal nucleotide DNA. The terminal nucleotide DNA is known and used to identify the target nucleotide. After the target nucleotide is identified, the target nucleotide may be compared to known single nucleotide polymorphisms to determine whether the target has a single nucleotide polymorphism. Alternatively, the target nucleotide and the probe DNA may be used to identify gene expression.

Methods of preparing a microarray having four probes 410, 420, 430, 440 are as disclosed previously for the method having match and mismatch probes 108, 119. The hybridization solution and method are as disclosed previously for the method having match and mismatch probes. The extension-ligation solution and method are as disclosed for the method having match and mismatch probes. The wash solution and method are as disclosed for the method having match and mismatch probes.

Referring to FIG. 5, in another embodiment of the present invention, a microarray-based single nucleotide polymorphism, sequencing, and gene expression assay method is provided. A target 500 is sequenced by preparing consecutive sequence probes comprising four probes for each base along the area of interest on the target. The probes are based on the complementary sequence 501. The first five bases of the target 500 are shown having four probes for each base. For the first base, A, four probes 502, 504, 506, 508 are shown having an optional spacer and attached to a microarray 501. For the second base, T, four probes 510, 512, 514, 516 are shown having an optional spacer and attached to a microarray 501. For the third base, T, four probes 520, 522, 524, 526 are shown having an optional spacer and attached to a microarray 501. For the fourth base, A, four probes 530, 532, 534, 536 are shown having an optional spacer and attached to a microarray 501. For the fifth base, T, four probes 540, 542, 544, 546 are shown having an optional spacer and attached to a microarray 501. Probe length is shown as 15 bases. Probe length is approximately 5 to approximately 100 nucleotides.

Hybridizing tagged target to the consecutive sequence probes and detection sequence DNA forms a plurality of consecutive sequence structures. The plurality of consecutive sequence structures is extended using an extension-ligation solution and an extension-ligation condition. The plurality of consecutive sequence structures having a terminal nucleotide complementary to a target nucleotide is ligated using the extension-ligation solution and the extension-ligation condition. Non-bound material is removed by using a washing solution and a washing method. A target nucleotide of the tagged targets is determined thus providing the sequence of the target from the consecutive sequence probes.

In an alternative embodiment, tagged target comprises tagged target RNA, detection sequence comprises detection sequence DNA, and probe comprises probe DNA. Preferably, the tagged target RNA comprises mRNA. The mRNA may be obtained from RNA embedded within paraffin. The probe DNA is place on a microarray. The probe DNA may be spotted on a microarray. The probed DNA may be synthesized in situ on a microarray. Preferably, the probe DNA is synthesized in situ on an electrode microarray. Preferably, the detection sequence DNA has a fluorescent tag. Preferably, the tagged target RNA and probe DNA have less than five internal mismatches when hybridized, and the tagged target RNA the detection sequence DNA have less than approximately five internal mismatches when hybridized.

The tagged target RNA and detection sequence DNA are hybridized on the microarray having probe DNA using a hybridizing solution and hybridizing method forming hybridized structure DNA/RNA. Preferably, the hybridizing solution comprises a plurality of tagged target RNA and a plurality of detection sequence DNA in a buffer solution comprising a T4 ligase buffer. Preferably, the hybridizing method comprises exposing the microarray to the hybridizing solution at approximately 45° C. for approximately one hour.

A single step extension and ligation reaction is performed using an extension ligation solution and method. The extension-ligation solution comprises water, buffer, dNTP, polymerase, and ligase. The method comprises incubation of the microarray exposed to the solution at approximately 37° C. for approximately one hour. The polymerase is a DNA polymerase. Preferably, the DNA polymerase is a reverse transcriptase. Preferably, the buffer comprises E. coli ligase buffer. Preferably, the ligase comprises E. coli ligase. Alternatively, the buffer comprises T4 ligase buffer and the ligase comprises T4 DNA ligase. Non-bound material is removed by washing. The wash solution is selected from the group consisting of buffer solution and base solution. Preferably, the buffer is a Tris buffer or a phosphate buffer, although other buffers are suitable. Preferably, the phosphate buffer is a phosphate buffered saline (PBS) having a pH of approximately 7 to 7.5. Preferably, the Tris buffer is a Tris-HCl having a pH of approximately 7 to 7.5. Preferably, the PBS buffer is 0.05×PBS buffer. Preferably, the base solution is an aqueous sodium hydroxide solution. Preferably, the concentration of base is approximately 0.01 to 5 molar. More preferably, the base concentration is approximately 0.05 to 1 molar. Most preferably, the base concentration is approximately 0.1 molar sodium hydroxide. The wash method comprises exposing the microarray to the wash solution at a temperature of approximately room temperature to approximately seventy degrees centigrade. Preferably, the wash method comprises exposing the microarray to the wash solution having base at approximately room temperature until sufficient non-bound material is removed to allow accurate reading of the microarray. Generally, washing approximately three times is sufficient.

The following examples are provided merely to explain, illustrate, and clarify the present invention and not to limit the scope or application of the present invention.

### EXAMPLE 1

A microarray-based SNP assay was performed on an electrode microarray by preparing a tagged target DNA from a genome of interest, the human ataxia telangiectasia mutated (ATM) gene. Four known mutations were detected in the human ATM gene from well characterized commercially available total genomic DNA samples. The samples were obtained from Coriell Cell Repositories and include the following: (1) Patient #1, NA02052; (2) Patient #2, NA 11261; (3) Patient #3, NA03189; and (4) Patient #4 (normal), NA13069. Patient #1 DNA contained one mutation in the ATM gene at nucleotide position 103 (C to T); Patient #2 DNA contained two mutations in the ATM gene at nucleotide positions 7327 (C to T) and 7926 (A to C); Patient #3 DNA contained one mutation in the ATM gene at nucleotide position 8266 (A to T); and Patient #4 contained no mutations in the ATM gene. A portion of each patient's genes having the location of the expected mutation(s) was selected and used as templates for PCR amplification to produce tagged target DNA for each patient sample for each location where the mutation is present.

Total genomic DNA samples from each patient were subjected to a two-stage PCR method for preparation of a solution of tagged target DNA for each mutation site. Thus, for each patient sample, four separate two-stage PCR preparations were performed to obtain tagged target DNA for each patient at each of the four locations where mutations can occur. For patient #1 sample, the DNA sequences of the target DNA comprised SEQ ID NO:7, 8, 9, and 10, and the complements thereof. For patient #2 sample, the DNA sequences of the target DNA comprised SEQ ID NO:11, 12, 13, and 10, and the complements thereof. For patient #3 sample, the DNA sequences of the target DNA comprised SEQ ID NO:11, 8, 9, and 14, and the complements thereof. For patient #4 sample, the DNA sequences of the target DNA comprised SEQ ID NO:11, 8, 9, and 10, and the complements thereof.

In the first PCR stage, a PCR solution was made comprising 67 microliters of purified water (Ambion Molecular Grade), 10 microliters of 10× polymerase buffer (New England BioLabs, Inc., #B9004S), 10 microliters of dimethylsulfoxide, 1 microliter of Taq DNA polymerase (New England BioLabs, Inc., #M0267S), 3 microliters of a 10 millimolar dNTP mix (New England BioLabs, Inc., #N0447S), 2 microliters of a 10 micromolar tagged specific forward primer (Integrated DNA Technologies, Inc.), 2 microliters of a 10 micromolar specific reverse primer (Integrated DNA Technologies, Inc.), and 5 microliters of genomic DNA at 20 nanograms per microliter. The forward primers used were as follows: SEQ ID NO:31 for ATM location 103; SEQ ID NO:33 for ATM location 7327; SEQ ID NO:35 for ATM location 7926; and SEQ ID NO:37 for ATM location 8266. The reverse primers used were as follows: SEQ ID NO:32 for ATM location 103; SEQ ID NO:34 for ATM location 7327; SEQ ID NO:36 for ATM location 7926; and SEQ ID NO:38 for ATM location 8266.

The reaction conditions comprised denaturation for 5 minutes at 94° C. and amplification for 30 cycles, wherein each cycle comprised denaturation for 30 second at 94° C., annealing for 30 sec at 55° C., and extension for 30 seconds at 72° C. A final extension was done for 10 minute at 72° C. to complete reactions. The PCR solution was then stored at approximately 0° C. or slightly lower in temperature until subjected to a second PCR stage; however, some samples were used immediately without storage and some were frozen for long-term storage. The PCR products for each patient from the first stage PCR were as follows: Patient #1—location 103 SEQ ID NO:15 and the complement thereof, location 7327 SEQ ID NO:16 and the complement thereof, location 7926 SEQ ID NO:17 and the complement thereof, and location 8266 SEQ ID NO:18 and the complement thereof; Patient #2—location 103 SEQ ID NO:19 and the complement thereof, location 7327 SEQ ID NO:20 and the complement thereof, location 7926 SEQ ID NO:21 and the complement thereof, and location 8266 SEQ ID NO:18 and the complement thereof; Patient #3-location 103 SEQ ID NO:19 and the complement thereof, location 7327 SEQ ID NO:16 and the complement thereof, location 7926 SEQ ID NO:17 and the complement thereof, and location 8266 SEQ ID NO:22 and the complement thereof; and Patient #4—location 103 SEQ ID NO:19 and the complement thereof, location 7327 SEQ ID NO:16 and the complement thereof, location 7926 SEQ ID NO:17 and the complement thereof, and location 8266 SEQ ID NO:18 and the complement thereof.

In the second PCR stage, a PCR solution was made comprising 67 microliters of purified water, 10 microliters of 10× polymerase buffer, 10 microliters of dimethylsulfoxide, 1 microliter of Taq DNA polymerase, 3 microliters of a 10 millimolar dNTP mix, 2 microliters of a 10 micromolar biotinylated T7 common forward primer, 2 microliters of a 10 micromolar specific reverse primer, and 5 microliters of amplification product from stage 1 that had been cleaned with a Qiagen QIAquick nucleotide removal kit (#28306) to remove primers from the first stage. The biotinylated forward primer used for all four ATM locations was SEQ ID NO:39. The reverse primers used were as follows: ATM location 103 used SEQ ID NO:32; ATM location 7327 used SEQ ID NO:34; ATM location 7926 used SEQ ID NO:36; and ATM location 8266 used SEQ ID NO:38.

The reaction conditions comprised denaturation for 5 minutes at 94° C. and amplification for 40 cycles, wherein each cycle comprised denaturation for 30 seconds at 94° C., annealing for 30 seconds at 55° C., and extension for 30 seconds at 72° C. A final extension was done for 10 minutes at 72° C. to complete reactions. The PCR solution was then stored at approximately 0° C. or slightly lower in temperature; however, some samples were used immediately without storage and some were frozen for long-term storage. The resulting product was purified with a Qiagen QIAquick Nucleotide Removal kit (#28306) and eluted with 100 microliters of purified water.

One hundred microliters of streptavidin magnetic beads (New England Biolabs #S 1420S) were washed three times using 2×PBS. The cleaned PCR product from above was brought to 2× PBS by adding 10×PBS and was mixed with the magnetic beads and allowed to incubate at room temperature for 15 minutes. This mixture was centrifuged at 6000 RPM for 1 minute in a microfuge and then the beads were washed twice with 2×PBS. After the last wash solution was removed, 20 microliters of a 0.1 molar NaOH solution was mixed with the beads and allowed to incubate at room temperature for 10 minutes. The beads were then centrifuged at 6000 RPM, and the supernatant was saved because it contained the tagged target DNA that was eluted by the NaOH. Twenty microliters of 0.1 molar NaOH was again mixed with the beads and allowed to incubate for 10 minutes. Finally, the beads were centrifuged; the supernatant was added to the previous 20 microliters; and the solution was neutralized with 20 microliters of 0.2 molar HCl and 7 microliters of 10×PBS. Again, the tagged target DNA was purified with a Qiagen QIAquick Nucleotide Removal kit (#28306) and eluted with 100 microliters of purified water (Ambion.)

The PCR products for each patient from the second stage PCR and after cleaning comprised the tagged target DNA and were as follows: Patient #1—location 103 SEQ ID NO:23 and the complement thereof, location 7327 SEQ ID NO:24 and the complement thereof, location 7926 SEQ ID NO:25 and the complement thereof, and location 8266 SEQ ID NO:26 and the complement thereof; Patient #2—location 103 SEQ ID NO:27 and the complement thereof, location 7327 SEQ ID NO:28 and the complement thereof, location 7926 SEQ ID NO:29 and the complement thereof, and location 8266 SEQ ID NO:26 and the complement thereof; Patient #3-location 103 SEQ ID NO:27 and the complement thereof, location 7327 SEQ ID NO:24 and the complement thereof, location 7926 SEQ ID NO:25 and the complement thereof, and location 8266 SEQ ID NO:30 and the complement thereof; and Patient #4—location 103 SEQ ID NO:27 and the complement thereof, location 7327 SEQ ID NO:24 and the complement thereof, location 7926 SEQ ID NO:25 and the complement thereof, and location 8266 SEQ ID NO:26 and the complement thereof. Each sequence from the second stage PCR was identical to the sequences from the first stage PCR except for an optional additional detection sequence complement DNA [SEQ ID NO:75 and the complement thereof] attached at the 5′ end.

The probe DNA was synthesized on the microarray device and did not have a 5′ phosphate. The probe DNA was phosphorylated using a solution of T4 polynucleotide kinase (PNK) in 175 microliters of purified water (Ambion) having 20 microliters of 10×PNK buffer, 2.0 microliters of 100 millimolar rATP (Promega, rATP, #E6011,) and 3 microliters (40 units) of PNK. The mixture was added to the microarray slide and the slide was incubated for 30 minutes at 37° C. to complete phosphorylation. Probe DNA on the electrode microarray was designed so that the predicted melting temperature (TM) was approximately 50° C. The probe DNA sequences for each SNP location were as follows: SNP location 103 SEQ ID NO:40, SNP location 7327 SEQ ID NO: 42, SNP location 7926 SEQ ID NO:44, and SNP location 8266 SEQ ID NO:46. To compare the present invention to the hybridization method, probes were synthesized on the microarray device having the SNP internal to the probes rather than terminal on the probes. The internal probe sequences for each SNP location were as follows: SNP location 103 SEQ ID NO:41, SNP location 7327 SEQ ID NO: 43, SNP location 7926 SEQ ID NO:45, and SNP location 8266 SEQ ID NO:47.

Optional DNA spacers of five, ten, and fifteen nucleotides in length were used for different electrode locations having the different probes thereon. The five nucleotide spacer was SEQ ID NO:48; the TEN nucleotide spacer was SEQ ID NO:49; the fifteen nucleotide spacer was SEQ ID NO:50. The probe DNA sequences for each SNP location having the optional five nucleotide spacer were as follows: SNP location 103 SEQ ID NO:51, SNP location 7327 SEQ ID NO: 53, SNP location 7926 SEQ ID NO:55, and SNP location 8266 SEQ ID NO:57. The internal probe sequences for each SNP location having the optional five nucleotide spacer were as follows: SNP location 103 SEQ ID NO:52, SNP location 7327 SEQ ID NO: 54, SNP location 7926 SEQ ID NO:56, and SNP location 8266 SEQ ID NO:58.

The probe DNA sequences for each SNP location having the optional ten nucleotide spacer were as follows: SNP location 103 SEQ ID NO:59, SNP location 7327 SEQ ID NO: 61, SNP location 7926 SEQ ID NO:63, and SNP location 8266 SEQ ID NO:65. The internal probe sequences for each SNP location having the optional ten nucleotide spacer were as follows: SNP location 103 SEQ ID NO:60, SNP location 7327 SEQ ID NO: 62, SNP location 7926 SEQ ID NO:64, and SNP location 8266 SEQ ID NO:66.

The probe DNA sequences for each SNP location having the optional fifteen nucleotide spacer were as follows: SNP location 103 SEQ ID NO:67, SNP location 7327 SEQ ID NO: 69, SNP location 7926 SEQ ID NO:71, and SNP location 8266 SEQ ID NO:73. The internal probe sequences for each SNP location having the optional fifteen nucleotide spacer were as follows: SNP location 103 SEQ ID NO:68, SNP location 7327 SEQ ID NO: 70, SNP location 7926 SEQ ID NO:72, and SNP location 8266 SEQ ID NO:74.

Each solution having the tagged target DNA was heated to 95° C. for 15 minutes and then placed on ice for storage. For each patient, the tagged target DNA corresponding to each SNP location were combined into one solution prior to contacting with a separate electrode microarray for each patient. Ten times T4 ligase buffer was added to the combined solution bring the solution to 1×, and detection sequence DNA was added to a concentration of 1 micromolar. This mixture was added to the slide hybridization chamber and incubated at 45° C. for 1 hour for hybridization of the tagged target DNA to the probe DNA and the detection sequence DNA to the tagged target DNA for each patient. Half of each electrode array was used for terminal SNP assay in accordance with the present invention and half was used for internal SNP hybridization assay to compare to the terminal SNP assay of the present invention.

After washing each electrode microarray twice with 2×PBS, a mixture of 155 microliter of purified water (Ambion,) 18 microliters of 10×E. coli ligase buffer (New England Biolabs, Inc.,) 3 microliters of 10 millimolar dNTP, 2 microliters (20 units) of Taq polymerase Stoffel fragment (Applied Biosystems, Amplitaq DNA Polymerase, Stoffel fragment, # N8080038; a reverse transcriptase such as Invitrogen SuperScript II Reverse Transcriptase #18064014 could also have been used,) and 2 microliters (20 units) of E. coli ligase (New England BioLabs, Inc. #M0205L) was added to the electrode microarray and was incubated at 37° C. for 1 hour. Finally, each microarray was washed 5 times with 0.05×PBS at 65° C. and viewed with a microarray slide scanner/reader.

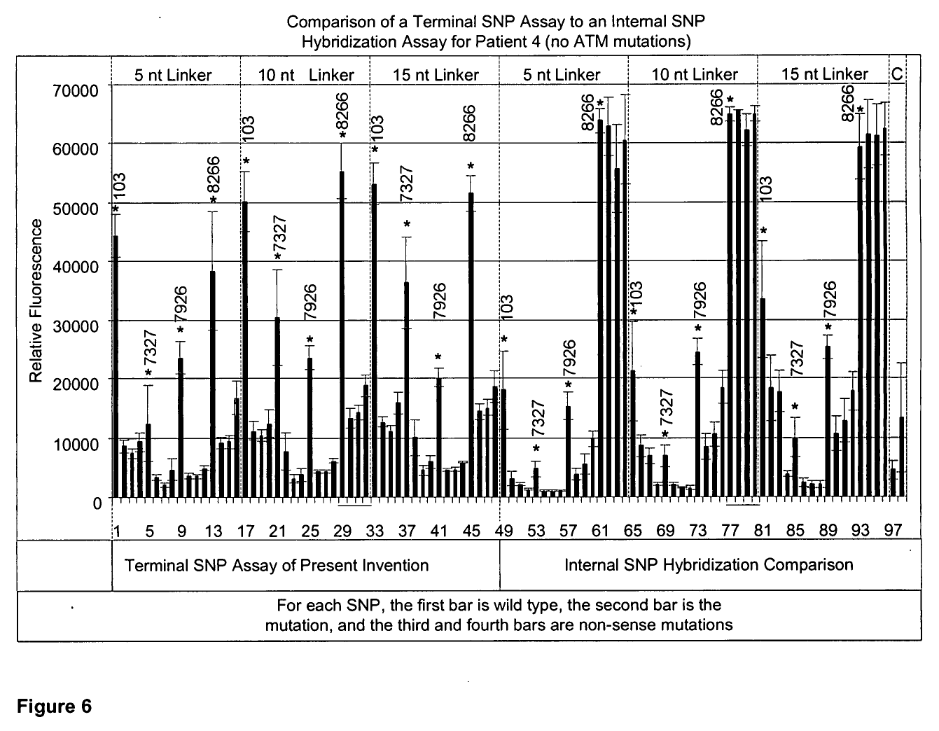

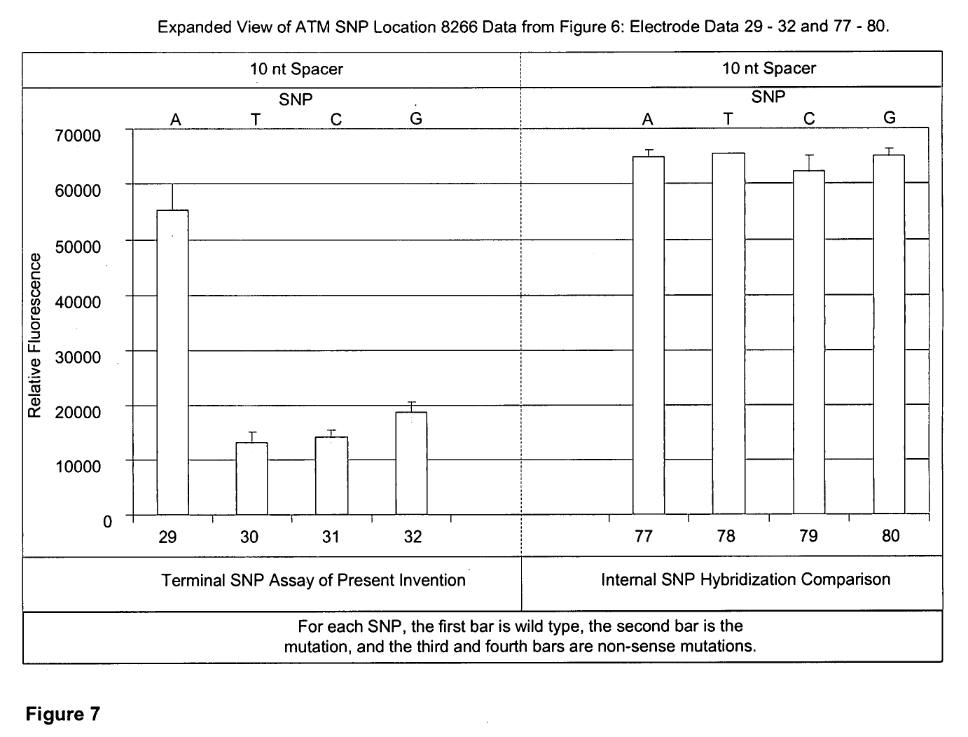

The results of the SNP assays are shown in FIGS. 6-13. FIG. 6 is a bar chart comparison of the microarray-based SNP assay of the present invention compared to SNP detection using hybridization to detect an internal SNP. The data is of the ATM gene of Patient #4, who has no mutations. Bars 61-64, 77-80, and 93-96 represent non-discriminate target hybridization to A/T-rich probes (28.6% G/C) that are 28 nucleotides in length while bars 53-56, 69-72, and 85-88 represent selective target hybridization to G/C-rich probes (60% G/C) that are only 15 nucleotides in length. In contrast, all predicted sequences for all targets analyzed were correctly determined with the combined extension/ligation terminal SNP assay of the present invention. Numbered bars represent the un-modified, expected genomic sequence, while the next 3 unnumbered bars represent the predicted/potential SNP followed by two nonsense mutations. The bars indicate a mean of eight replicates, and the lines indicate plus or minus one standard deviation. Bars 97 (5 nucleotide spacer) and 98 (10 nucleotide spacer) represent controls for completeness of washing and quality of synthesized probe DNA.

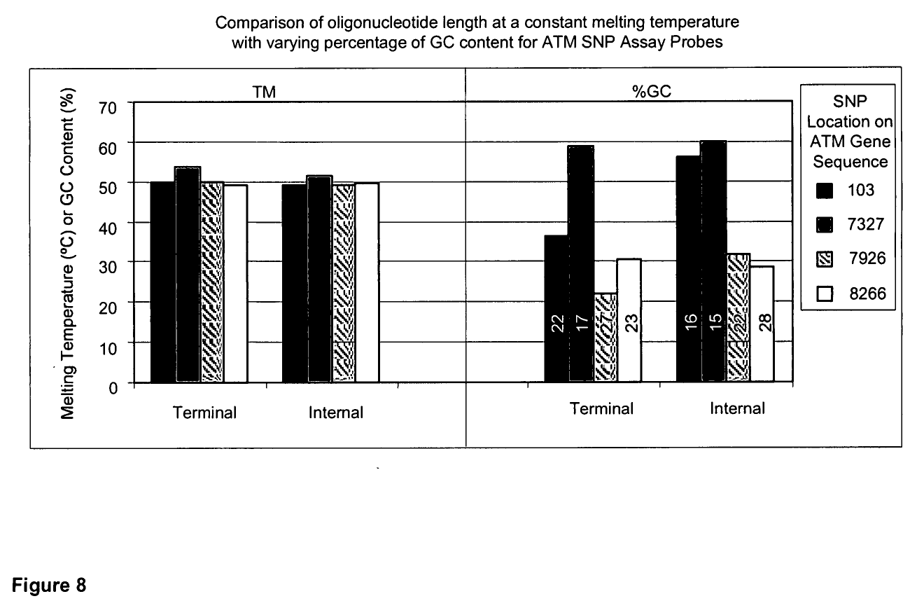

Refering to FIGS. 6 and 7, the microarray-based SNP assay of the present invention (Terminal SNP Assay) is an improvement over the traditional hybridization assay (Internal SNP Hybridization Assay) because the present invention is able to distinguish whether a SNP is present, whereas the traditional hybridization assay is not able to distinguish whether a SNP is present under all conditions. The reduced sensitivity of the standard hybridization assay is due to probe sequence compositions that are A/T-rich, that, when adjusted in length to a standard melting temperature (TM), will be greater in length than probes with GC-rich sequences and will hybridize to the probes at certain temperatures, even when containing an internal mismatch. The right panel of FIG. 6 and of FIG. 7 illustrates the problem with the internal SNP method as shown by the overlap of bars 61-64 (5 nt spacer), 77-80 (10 nt spacer), and 93-96 (nt spacer). A/T-rich probes, represented by bars 61-64, 77-80, and 93-96, were 28 nt in length while G/C-rich probes, represented by bars 53-56, 69-72, and 85-88, were only 15 nucleotides in length when adjusted to a TM of approximately 50° C. at shown in FIG. 8.

When the tagged targets hybridized to the probes at a constant temperature, 45° C. in this case, the mutation could be discriminated in the G/C-rich probes, but not in the A/T-rich probes. The terminal SNP assay is not affected by probe sequence length as long as TMs (melting temperatures) are approximately standardized. The microarray-based assay of the present invention is also an improvement over the traditional ligation SNP assay where a labeled oligonucleotide anneales to the target sequence adjacent to the probe and ligase is used to distinguish match and mismatch. The traditional assay requires novel labeled oligonucleotides for each SNP while the approach detailed here utilizes unique oligonucleotides that are unlabeled. All labeled oligonucleotides, either biotin or fluorescent, are common to all assays. This allows for the amplification of target sequences from sample genomic DNA with relatively inexpensive unlabeled primers, and a reamplification step that utilizes common labeled primers (biotinylated or fluorescent).

Referring to FIG. 8, DNA probes adjusted for a constant melting temperature (TM; left panel) show an inverse relationship between the percentage of G/C content (right panel) and probe length (numbers on bars). Thus, a probe with a high G/C content will be short (15 nt for internal 7327) while a probe with a high A/T content will be relatively longer (28 nt for internal 8266) and under certain hybridization conditions, will not discriminate a mismatch with hybridization alone.

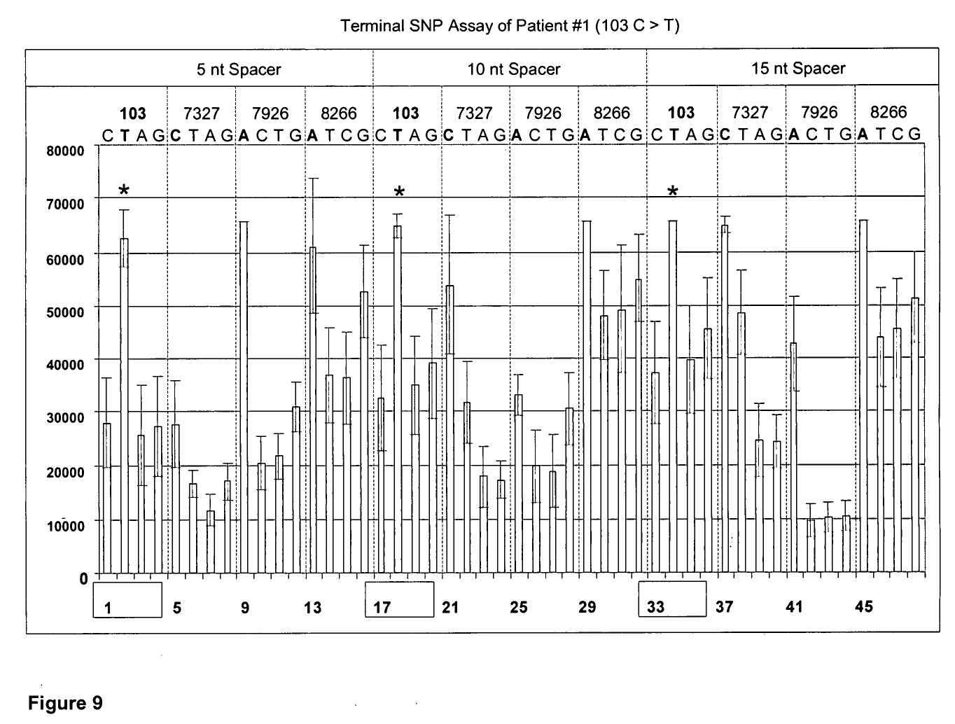

FIGS. 9 through 12 provide a bar chart representating SNP data for each of the four patients. The optional nucleotide spacer was used on all probes, and the length was 5, 10 or 15 nucleotides [SEQ ID NO:51, 52, and 53]. The four SNP locations for each patient are shown on each figure. The vertical axis is relative fluorescence, and the horizontal axis is an arbitrary number for the terminal DNA for the respective probes for each SNP. The bars are the mean of eight electrode locations, and the error bars represent the standard deviation of the eight electrode locations. The first probe in each series of four probes is wild type. The second probe is the SNP. The third and fourth probes are non-sense mutations. Also shown are the results of the inclusion of a 5, 10, or 15 nucleotide spacer between the electrode microarray and the sequence of interest. The probes representing sequences of interest for each SNP have been boxed below, and the terminal nucleotide for each probe is shown above each graph.

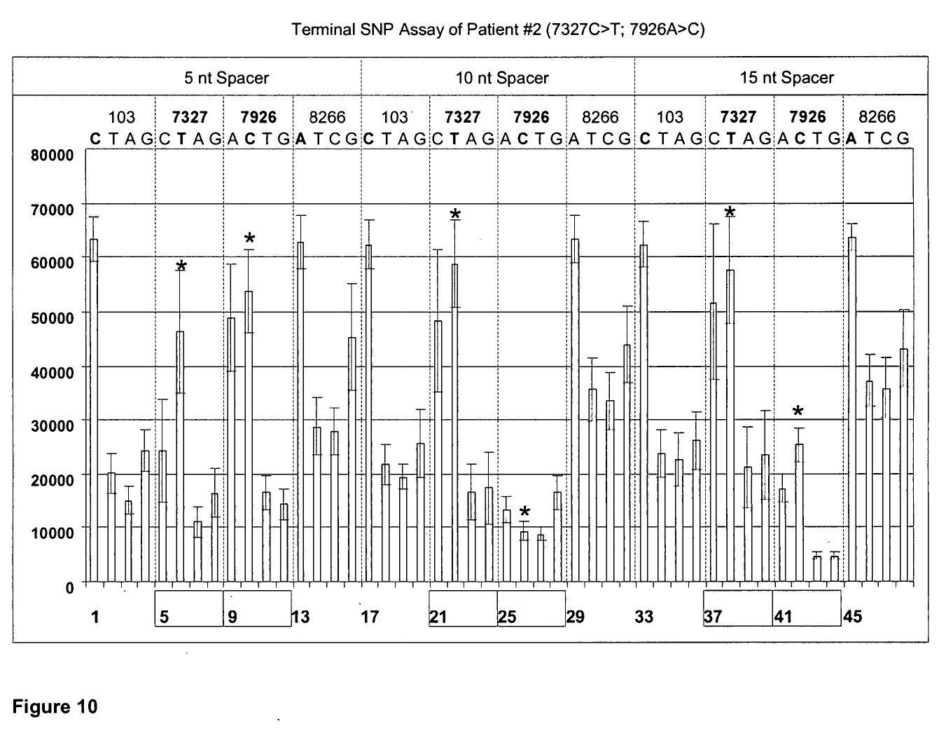

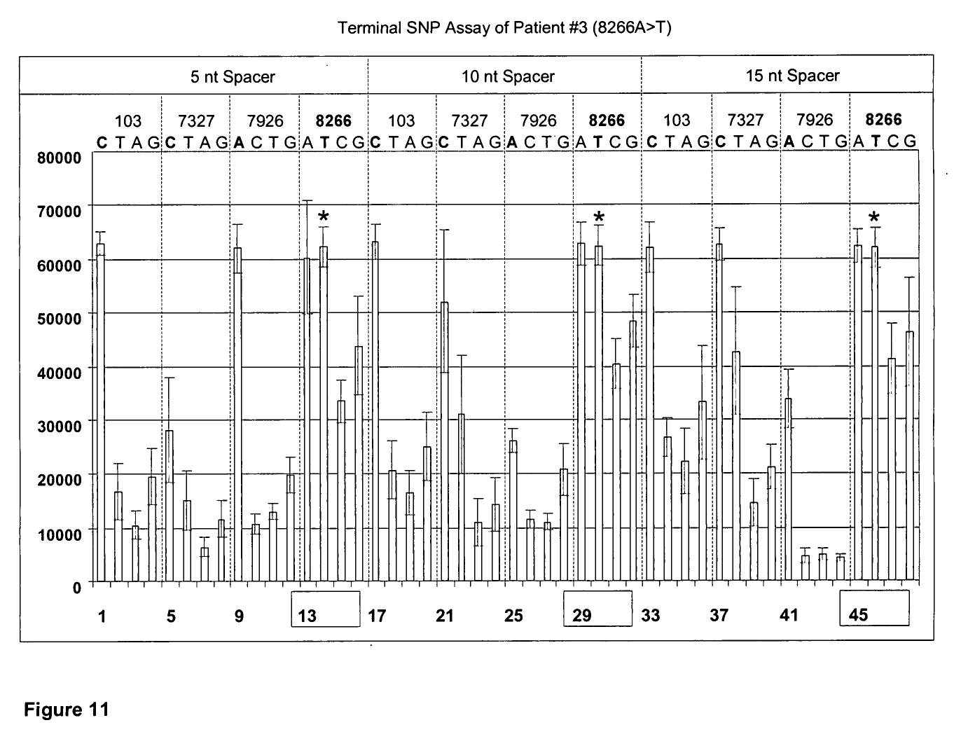

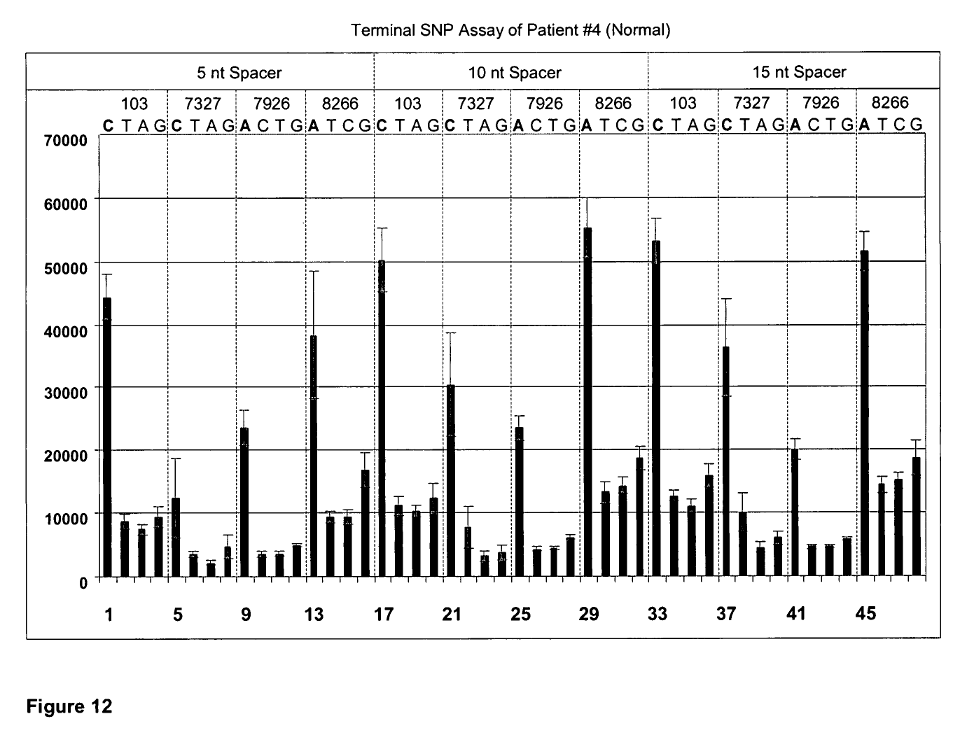

FIG. 9 is for Patient #1 and shows that the SNP (C>T) at location 103 is detected at each spacer length as noted by the star on the bars. FIG. 10 is for Patient #2 and shows that the two SNP (C>T; A>C) at locations 7327 and 7926 are detected at each spacer length as noted by the star on the bars. FIG. 11 is for Patient #3 and shows that the SNP (A>T) at location 8266 are detected at each spacer length as noted by the star on the bars. FIG. 12 is for Patient #4 and there are no SNP detected. In all cases, the expected SNP was detected. Patient #1 was found to be homozygous for the 103 C to T mutation and normal for the other 3 potential mutation sites; Patient #2 was found to be heterozygous for the 7327 C to T mutation and for the 7926 A to C mutation; and Patient #3 was also found to be heterozygous for the 8266 A to T mutation site and normal for other sites. As expected, normal donor 4 showed the wild type sequence for all potential SNP sites examined.

### EXAMPLE 2

This example provides an analysis of gene expression on an oligonucleotide microarray. This method provides for the direct use of messenger RNA (mRNA) in hybridization studies without the need for use of a label on the mRNA because it has a polydeoxyadenylate as a natural label or tag on the 3′ end. The mRNA is the tagged target. Probes are attached to a microarray by in situ electrochemical synthesis. The probes are phosphorylated using a kinasing solution comprising a mixture of 174 microliters purified water, 20 microliters PNK buffer, 2 microliters 100 millimolar rATP, and 4 microliters PNK. The solution is contacted to the microarray, which is incubated for 30 minutes at 37° C. while in contact with the solution.

To prepare the mRNA solution for hybridization of the mRNA to probes on the microarray, the mRNA is added to purified water and incubated at 70° C. for 10 minutes. After incubation, the mRNA solution is placed on ice. To the mRNA solution is added 10×T4 ligase buffer to a final concentration of 1×T4 ligase buffer (18 microliters 10× buffer in 160 microliters mRNA) (New England Biolabs) and 2 microliters of labeled oligonucleotide dT-30-mer (detection sequences) to a final concentration of approximately 1 micromolar. The label is a fluorescent label, such as a Cy3 or Cy5. This mixture of the mRNA and the labeled oligonucleotide is added to the hybridization chamber of the microarray having the probes and incubated for 1 to 18 hr at 40 to 45° C. Alternatively, the labeled dT-30-mer oligonucleotide is added separately from the mRNA solution as an additional step after the step of hybridization of the mRNA to the probes. If added separately, the labeled oligonucleotide is incubated at 40° C. for 30 minutes to allow hybridization of the labeled oligonucleotide to the mRNA.

After hybridization of the mRNA to the probes and the labeled oligonucleotide to the mRNA, the microarray is washed with 1×T4 ligase buffer when T4 ligase is used in the extension-ligation solution. Alternatively, the microarray is washed with 1×E. coli ligase buffer when E. coli ligase is used in the extension-ligation solution. The extension/ligation solution is contacted to the microarray to extend the labeled oligonucleotide to the terminal base of the probes and to ligate the labeled oligonucleotide to the probes when the terminal base on the probes is complementary to the base on the hybridized mRNA. The extension-ligation solution is comprised of 154 microliters of purified water, 18 microliters E. coli Ligase buffer, 4 microliters 10 mM dNTP mix, 2 microliters of SuperScript II reverse transcriptase (Invitrogen), and 2 microliters of E. coli DNA ligase. Alternatively, the extension-ligation solution is comprised of 154 microliters of purified water, 18 microliters T4 ligase buffer, 4 microliters 10 mM dNTP mix, 2 microliters of SuperScript II reverse transcriptase (Invitrogen), and 2 microliters of T4 DNA ligase. The extension-ligation solution is contacted to the microarray and incubated for 1 hour at 37° C. After incubation, the microarray is washed five times using purified water at 70° C. Alternatively, the microarray is washed two times using 0.1 N NaOH at room temperature or until the extended and unligated labeled oligonucleotide dT-30-mer (detection sequences) is removed. After washing, the microarray is placed in a fluorescent imaging devices to scan for the fluorescent label at the appropriate wavelength. Cy3 is scanned at wavelength 595 nanometers. Cy5 is scanned at wavelength 685 nanometers. Washing substantially removes mRNA (target) and labeled oligonucleotide dT-30-mer (detection sequences) except for the label that has been ligated to the appropriate probe by the extension and ligation step.

We claim:

1. A microarray-based single nucleotide polymorphism, sequencing, and gene expression assay method comprising:
(a) providing a microarray having a plurality of probes;
(b) forming a plurality of hybridized structures on the microarray, wherein each hybridized structure is formed by contacting the microarray under a hybridizing condition to a hybridizing solution comprising a plurality of tagged targets and a plurality of detection sequences, wherein each hybridized structure comprises one tagged target hybridized to one probe and to one detection sequence;
(c) extending each hybridized structure using an extension-ligation solution;
(d) ligating each hybridized structure having a terminal nucleotide that is complementary to a target nucleotide using the extension-ligation solution;
(e) removing non-bound material by washing the microarray using a wash solution; and
(f) identifying the target nucleotide and a hybridized sequence from the hybridized structures having ligation.

2. The method of claim 1, wherein the plurality of probes is selected from the group consisting of probe DNA, probe RNA, and combinations thereof.

3. The method of claim 1, wherein the plurality of probes is attached to the microarray by a spacer.

4. The method of claim 1, wherein the plurality of tagged targets is selected from the group consisting of tagged target DNA, tagged target RNA, and combinations thereof.

5. The method of claim 3, wherein the tagged target DNA is cDNA.

6. The method of claim 3, wherein the tagged target RNA is mRNA.

7. The method of claim 1, wherein the plurality of tagged targets is first amplified.

8. The method of claim 1, wherein the plurality of detection sequences is selected from the group consisting of a detection sequence DNA, a detection sequence RNA, and combinations thereof.

9. The method of claim 1, wherein the plurality of detection sequences has a fluorescent tag.

10. The method of claim 1, wherein the plurality of tagged targets and the plurality of probes have less than approximately five internal mismatches when hybridized, and the plurality of tagged targets and the plurality of detection sequences have less than approximately five internal mismatches when hybridized.

11. The method of claim 1, wherein the hybridizing solution comprises a plurality of tagged targets and a plurality of detection sequences in a buffer solution comprising a 1×T4 ligase buffer, and the hybridizing condition comprises approximately 45° C. for approximately one hour.

12. The method of claim 1, wherein the extension-ligation solution comprises a formulation comprising water, buffer, triphosphate mix, polymerase, and ligase.

13. The method of claim 12, wherein the polymerase is selected from the group consisting of DNA polymerase and RNA polymerase, and combinations thereof.

14. The method of claim 12, wherein the polymerase is selected from the group consisting of Taq polymerase Stoffel fragment, a reverse transcriptase, E. coli DNA polymerase, Klenow fragment polymerase, T7 RNA polymerase, T3 RNA polymerase, viral replicase, and SP6 RNA polymerase, and combinations thereof.

15. The method of claim 12, wherein the buffer is selected from the group consisting of T4 DNA ligase buffer and T4 RNA ligase buffer, and combinations thereof.

16. The method of claim 12, wherein the ligase is selected from the group consisting of E. coli DNA ligase, T4 DNA ligase, and T4 RNA ligase, and combinations thereof.

17. The method of claim 12, wherein the triphosphate mix is selected from the group consisting of dNTP and rNTP.

18. A microarray-based single nucleotide polymorphism, sequencing, and gene expression assay method comprising:
(a) providing a microarray having a plurality of probe DNA;
(b) forming a plurality of hybridized structure DNA on the microarray, wherein each hybridized structure DNA is formed by contacting the microarray under a hybridizing condition to a hybridizing solution comprising a plurality of tagged target DNA and a plurality of detection sequence DNA, wherein each hybridized structure DNA comprises one tagged target DNA hybridized to one probe DNA and to one detection sequence DNA;
(c) extending each hybridized structure DNA using an extension-ligation solution;
(d) ligating each hybridized structure DNA having a terminal nucleotide DNA that is complementary to a target nucleotide DNA using the extension-ligation solution;
(e) removing non-bound material by washing the microarray using a wash solution; and
(f) identifying the target nucleotide DNA and a hybridized sequence DNA from the hybridized structures having ligation.

19. The method of claim 18, wherein the plurality of probe DNA is attached to the microarray by a spacer.

20. The method of claim 18, wherein the tagged target DNA is a cDNA.

21. The method of claim 18, wherein the tagged target DNA is first amplified.

22. The method of claim 21, wherein the amplification is by PCR.

23. The method of claim 18, wherein the plurality of detection sequence DNA has a fluorescent tag.

24. The method of claim 18, wherein the plurality of tagged target DNA and the plurality of probe DNA have less than five internal mismatches when hybridized, and the plurality of tagged target DNA and the plurality of detection sequence DNA have less than approximately five internal mismatches when hybridized.

25. The method of claim 18, the hybridizing solution comprises a plurality of tagged target DNA and a plurality of detection sequence DNA in a buffer solution comprising a 1×T4 ligase buffer, and the hybridizing condition comprises approximately 45° C. for approximately one hour.

26. The method of claim 18, wherein the extension-ligation solution comprises a formulation comprising water, buffer, dNTP, polymerase, and ligase.

27. The method of claim 26, wherein the polymerase is a DNA polymerase.

28. The method of claim 27, wherein the DNA polymerase is selected from the group consisting of Taq polymerase Stoffel fragment, a reverse transcriptase, E. coli polymerase, and Klenow fragment polymerase, and combinations thereof.

29. The method of claim 26, wherein the buffer comprises E. coli ligase buffer, and the ligase comprises E. coli ligase.

30. The method of claim 34, wherein the buffer comprises T4 ligase buffer and the ligase comprises T4 DNA ligase.

31. The method of claim 18, wherein the plurality of probe DNA comprises a plurality of match probe DNA and a plurality of mismatch probe DNA, and the plurality of hybridized structure DNA comprises a plurality of match structures and a plurality of mismatch structures.

32. The method of claim 18, wherein the plurality of probe DNA comprises a plurality of set probes, and the plurality of hybridized structure DNA comprises a plurality of set structures.

33. The method of claim 18, wherein the plurality of probe DNA comprises a plurality of consecutive sequence probes, and the plurality of hybridized structure DNA comprises a plurality of consecutive sequence structures.

34. The method of claim 18, wherein the plurality of probe DNA comprises a plurality of gene expression probes, and the plurality of hybridized structure DNA comprises a plurality of gene expression structures.

35. A microarray-based single nucleotide polymorphism, sequencing, and gene expression assay method comprising:
(a) providing a microarray having a plurality of probe DNA;
(b) forming a plurality of hybridized structure DNA/RNA on the microarray, wherein each hybridized structure DNA/RNA is formed by contacting the microarray under a hybridizing condition to a hybridizing solution comprising a plurality of tagged target RNA and a plurality of detection sequence DNA, wherein each hybridized structure DNA/RNA comprises one tagged target RNA hybridized to one probe DNA and to one detection sequence DNA;
(c) extending each hybridized structure DNA/RNA using an extension-ligation solution and an extension-ligation condition;
(d) ligating each hybridized structure DNA/RNA having a terminal nucleotide DNA that is complementary to a target nucleotide RNA using the extension-ligation solution and the extension-ligation condition;
(e) removing non-bound material by washing the microarray using a wash solution and a wash method; and
(f) identifying the target nucleotide RNA and a hybridized sequence RNA from the hybridized structures having ligation.

36. The method of claim 35, wherein the plurality of probes is attached to the microarray by a spacer.

37. The method of claim 35, wherein the tagged target RNA is a mRNA.

38. The method of claim 35, wherein the plurality of detection sequence DNA has a fluorescent tag.

39. The method of claim 35, wherein the plurality of tagged target RNA and the plurality of probe DNA have less than five internal mismatches when hybridized, and the plurality of tagged target RNA and the plurality of detection sequence DNA have less than approximately five internal mismatches when hybridized.

40. The method of claim 35, the hybridizing solution comprises a plurality of tagged target RNA and a plurality of detection sequence DNA in a buffer solution comprising a 1×T4 ligase buffer, and the hybridizing condition comprises approximately 45° C. for approximately one hour.

41. The method of claim 35, wherein the extension-ligation solution comprises water, buffer, dNTP, polymerase.

42. The method of claim 41, wherein the polymerase is a reverse transcriptase.

43. The method of claim 41, wherein the buffer comprises E. coli ligase buffer, and the ligase comprises E. Coli ligase.

44. The method of claim 41, wherein the buffer comprises T4 ligase buffer and the ligase comprises T4 DNA ligase.


SEQUENCE LISTING (78 sequences)

SEQ ID NO: 1 - DNA, 9 bp, Artificial Sequence
  An arbitraray example of a small DNA sequence that is considered non-mutated
atatgcact

SEQ ID NO: 2 - DNA, 9 bp, Artificial Sequence
  An arbitraray example of a small DNA sequence that is a mutated form of SEQ ID NO. 1
atattcact

SEQ ID NO: 3 - DNA, 9 bp, Artificial Sequence
  An arbitraray example of a small DNA sequence that is considered wild-type
atatgccgt

SEQ ID NO: 4 - DNA, 9 bp, Artificial Sequence
  An arbitraray example of a small DNA sequence that is considered a mutation of SEQ ID NO. 3
atacgccgt

SEQ ID NO: 5 - DNA, 19 bp, Artificial Sequence
  A T7 -based DNA sequence complementary to a primer
taatacgactcactatagg

SEQ ID NO: 6 - DNA, 25 bp, Artificial Sequence
  A T7 -based DNA sequence complementary to a primer
gcatcctaatacgactcactatagg

SEQ ID NO: 7 - DNA, 138 bp, Homo Sapien
tttccttgaaataagtgtgattagtaacccattattatttcctttttattttcagaaagaagttgagaaatttaagcgcctgatttgagatcctgaaacaattaaacatctagatcggcattcagattccaaacaagg

SEQ ID NO: 8 - DNA, 147 bp, Homo Sapien
gttgtatatcatgtgtgattttgtagttctgttaaagttcatggcttttgtgttttaccttaattattctatgcaagatacacagtaaaggttcagcgagagctggagttggatgaattagccctgcgtgcactgaaagaggatcgt

SEQ ID NO: 9 - DNA, 145 bp, Homo Sapien
aagtaggagacctcagatggtcagaagtgttgaggcactttgtgatgcttatattatattagcaaacttagatgccactcagtggaagactcagagaagtatgttttttttaaagaagaaacgttactttcttgctgtgttactc

SEQ ID NO: 10 - DNA, 128 bp, Homo Sapien
aatacattactgcagagaaacacggaaactaggaagaggaaattaactatctgtacttataaggtaactatttgtacttctgttagttcaccaaaaacatataaaagatgccatttggttgggtgaag

SEQ ID NO: 11 - DNA, 138 bp, Homo Sapien
tttccttgaaataagtgtgattagtaacccattattatttcctttttattttcagaaagaagttgagaaatttaagcgcctgattcgagatcctgaaacaattaaacatctagatcggcattcagattccaaacaagg

SEQ ID NO: 12 - DNA, 147 bp, Homo Sapien
gttgtatatcatgtgtgattttgtagttctgttaaagttcatggcttttgtgttttaccttaattattctatgcaagatacacagtaaaggttcagtgagagctggagttggatgaattagccctgcgtgcactgaaagaggatcgt

SEQ ID NO: 13 - DNA, 145 bp, Homo Sapien
aagtaggagacctcagatggtcagaagtgttgaggcactttgtgatgcttatattatattagcaaacttagatgccactcagtggaagactcagagcagtatgttttttttaaagaagaaacgttactttcttgctgtgttactc

SEQ ID NO: 14 - DNA, 128 bp, Homo Sapien
aatacattactgcagagaaacacggaaactaggaagaggaaattaactatctgtacttattaggtaactatttgtacttctgttagttcaccaaaaacatataaaagatgccatttggttgggtgaag

SEQ ID NO: 15 - DNA, 157 bp, Artificial Sequence
  Homo sapien having a T7 primer tag for hybridization
taatacgactcactataggtttccttgaaataagtgtgattagtaacccattattatttcctttttattttcagaaagaagttgagaaatttaagcgcctgatttgagatcctgaaacaattaaacatctagatcggcattcagattccaaacaagg

SEQ ID NO: 16 - DNA, 166 bp, Artificial Sequence
  Homo sapien having a T7 primer tag for hybridization
taatacgactcactatagggttgtatatcatgtgtgattttgtagttctgttaaagttcatggcttttgtgttttaccttaattattctatgcaagatacacagtaaaggttcagcgagagctggagttggatgaattagccctgcgtgcactgaaagaggatcgt

SEQ ID NO: 17 - DNA, 164 bp, Artificial Sequence
  Homo sapien having a T7 primer tag for hybridization
taatacgactcactataggaagtaggagacctcagatggtcagaagtgttgaggcactttgtgatgcttatattatattagcaaacttagatgccactcagtggaagactcagagaagtatgttttttttaaagaagaaacgttactttcttgctgtgttactc

SEQ ID NO: 18 - DNA, 147 bp, Artificial Sequence
  Homo sapien having a T7 primer tag for hybridization
taatacgactcactataggaatacattactgcagagaaacacggaaactaggaagaggaaattaactatctgtacttataaggtaactatttgtacttctgttagttcaccaaaaacatataaaagatgccatttggttgggtgaag

SEQ ID NO: 19 - DNA, 157 bp, Artificial Sequence
  Homo sapien having a T7 primer tag for hybridization
taatacgactcactataggtttccttgaaataagtgtgattagtaacccattattatttcctttttattttcagaaagaagttgagaaatttaagcgcctgattcgagatcctgaaacaattaaacatctagatcggcattcagattccaaacaagg

SEQ ID NO: 20 - DNA, 166 bp, Artificial Sequence
  Homo sapien having a T7 primer tag for hybridization
taatacgactcactatagggttgtatatcatgtgtgattttgtagttctgttaaagttcatggcttttgtgttttaccttaattattctatgcaagatacacagtaaaggttcagtgagagctggagttggatgaattagccctgcgtgcactgaaagaggatcgt

SEQ ID NO: 21 - DNA, 164 bp, Artificial Sequence
  Homo sapien having a T7 primer tag for hybridization
taatacgactcactataggaagtaggagacctcagatggtcagaagtgttgaggcactttgtgatgcttatattatattagcaaacttagatgccactcagtggaagactcagagcagtatgttttttttaaagaagaaacgttactttcttgctgtgttactc

SEQ ID NO: 22 - DNA, 147 bp, Artificial Sequence
  Homo sapien having a T7 primer tag for hybridization
taatacgactcactataggaatacattactgcagagaaacacggaaactaggaagaggaaattaactatctgtacttattaggtaactatttgtacttctgttagttcaccaaaaacatataaaagatgccatttggttgggtgaag

SEQ ID NO: 23 - DNA, 163 bp, Artificial Sequence
  Homo sapien having a T7 primer tag for hybridization
gcatcctaatacgactcactataggtttccttgaaataagtgtgattagtaacccattattatttcctttttattttcagaaagaagttgagaaatttaagcgcctgatttgagatcctgaaacaattaaacatctagatcggcattcagattccaaacaagg

SEQ ID NO: 24 - DNA, 172 bp, Artificial Sequence
  Homo sapien having a T7 primer tag for hybridization
gcatcctaatacgactcactatagggttgtatatcatgtgtgattttgtagttctgttaaagttcatggcttttgtgttttaccttaattattctatgcaagatacacagtaaaggttcagcgagagctggagttggatgaattagccctgcgtgcactgaaagaggatcgt

SEQ ID NO: 25 - DNA, 170 bp, Artificial Sequence
  Homo sapien having a T7 primer tag for hybridization
gcatcctaatacgactcactataggaagtaggagacctcagatggtcagaagtgttgaggcactttgtgatgcttatattatattagcaaacttagatgccactcagtggaagactcagagaagtatgttttttttaaagaagaaacgttactttcttgctgtgttactc

SEQ ID NO: 26 - DNA, 153 bp, Artificial Sequence
  Homo sapien having a T7 primer tag for hybridization
gcatcctaatacgactcactataggaatacattactgcagagaaacacggaaactaggaagaggaaattaactatctgtacttataaggtaactatttgtacttctgttagttcaccaaaaacatataaaagatgccatttggttgggtgaag

SEQ ID NO: 27 - DNA, 163 bp, Artificial Sequence
  Homo sapien having a T7 primer tag for hybridization
gcatcctaatacgactcactataggtttccttgaaataagtgtgattagtaacccattattatttcctttttattttcagaaagaagttgagaaatttaagcgcctgattcgagatcctgaaacaattaaacatctagatcggcattcagattccaaacaagg

SEQ ID NO: 28 - DNA, 172 bp, Artificial Sequence
  Homo sapien having a T7 primer tag for hybridization
gcatcctaatacgactcactatagggttgtatatcatgtgtgattttgtagttctgttaaagttcatggcttttgtgttttaccttaattattctatgcaagatacacagtaaaggttcagtgagagctggagttggatgaattagccctgcgtgcactgaaagaggatcgt

SEQ ID NO: 29 - DNA, 170 bp, Artificial Sequence
  Homo sapien having a T7 primer tag for hybridization
gcatcctaatacgactcactataggaagtaggagacctcagatggtcagaagtgttgaggcactttgtgatgcttatattatattagcaaacttagatgccactcagtggaagactcagagcagtatgttttttttaaagaagaaacgttactttcttgctgtgttactc

SEQ ID NO: 30 - DNA, 153 bp, Artificial Sequence
  Homo sapien having a T7 primer tag for hybridization
gcatcctaatacgactcactataggaatacattactgcagagaaacacggaaactaggaagaggaaattaactatctgtacttattaggtaactatttgtacttctgttagttcaccaaaaacatataaaagatgccatttggttgggtgaag

SEQ ID NO: 31 - DNA, 48 bp, Artificial Sequence
  Forward primer for PCR
taatacgactcactataggtttccttgaaataagtgtgattagtaacc

SEQ ID NO: 32 - DNA, 23 bp, Artificial Sequence
  Reverse primer for PCR
ccttgtttggaatctgaatgccg

SEQ ID NO: 33 - DNA, 50 bp, Artificial Sequence
  Forward primer for PCR
taatacgactcactatagggttgtatatcatgtgtgattttgtagttctg

SEQ ID NO: 34 - DNA, 24 bp, Artificial Sequence
  Reverse primer for PCR
acgatcctctttcagtgcacgcag

SEQ ID NO: 35 - DNA, 41 bp, Artificial Sequence
  Forward primer for PCR
taatacgactcactataggaagtaggagacctcagatggtc

SEQ ID NO: 36 - DNA, 24 bp, Artificial Sequence
  Reverse primer for PCR
gagtaacacagcaagaaagtaacg

SEQ ID NO: 37 - DNA, 44 bp, Artificial Sequence
  Forward primer for PCR
taatacgactcactataggaatacattactgcagagaaacacgg

SEQ ID NO: 38 - DNA, 22 bp, Artificial Sequence
  Reverse primer for PCR
cttcacccaaccaaatggcatc

SEQ ID NO: 39 - DNA, 54 bp, Artificial Sequence
  Second stage primer for PCR
gcatcctaatacgactcactataggtttccttgaaataagtgtgattagtaacc

SEQ ID NO: 40 - DNA, 56 bp, Artificial Sequence
  Primer sequence for location 7327
gcatcctaatacgactcactatagggttgtatatcatgtgtgattttgtagttctg

SEQ ID NO: 41 - DNA, 47 bp, Artificial Sequence
  Primer for location 7926
gcatcctaatacgactcactataggaagtaggagacctcagatggtc

SEQ ID NO: 42 - DNA, 50 bp, Artificial Sequence
  Primer for location 8266
gcatcctaatacgactcactataggaatacattactgcagagaaacacgg

SEQ ID NO: 43 - DNA, 22 bp, Homo Sapien
cgagatcctgaaacaattaaac

SEQ ID NO: 44 - DNA, 16 bp, Homo Sapien
gcctgattcgagatcc

SEQ ID NO: 45 - DNA, 17 bp, Homo Sapien
cgagagctggagttgga

SEQ ID NO: 46 - DNA, 15 bp, Homo Sapien
ggttcagcgagagct

SEQ ID NO: 47 - DNA, 27 bp, Homo Sapien
aagtatgttttttttaaagaagaaacg

SEQ ID NO: 48 - DNA, 22 bp, Homo Sapien
aagactcagagaagtatgtttt

SEQ ID NO: 49 - DNA, 23 bp, Homo Sapien
aaggtaactatttgtacttctgt

SEQ ID NO: 50 - DNA, 28 bp, Homo Sapien
ctatctgtacttataaggtaactatttg

SEQ ID NO: 51 - DNA, 5 bp, Artificial Sequence
  DNA spacer/linker for probe
agcct

SEQ ID NO: 52 - DNA, 10 bp, Artificial Sequence
  DNA spacer/linker for probe
agcctactgt

SEQ ID NO: 53 - DNA, 15 bp, Artificial Sequence
  DNA spacer/linker for probe
agcctactgatgagt

SEQ ID NO: 54 - DNA, 27 bp, Artificial Sequence
  Homo sapien probe having a spacer/linker
agcctcgagatcctgaaacaattaaac

SEQ ID NO: 55 - DNA, 21 bp, Artificial Sequence
  Homo sapien probe having a spacer/linker
agcctgcctgattcgagatcc

SEQ ID NO: 56 - DNA, 22 bp, Artificial Sequence
  Homo sapien probe having a spacer/linker
agcctcgagagctggagttgga

SEQ ID NO: 57 - DNA, 20 bp, Artificial Sequence
  Homo sapien probe having a spacer/linker
agcctggttcagcgagagct

SEQ ID NO: 58 - DNA, 32 bp, Artificial Sequence
  Homo sapien probe having a spacer/linker
agcctaagtatgttttttttaaagaagaaacg

SEQ ID NO: 59 - DNA, 27 bp, Artificial Sequence
  Homo sapien probe having a spacer/linker
agcctaagactcagagaagtatgtttt

SEQ ID NO: 60 - DNA, 28 bp, Artificial Sequence
  Homo sapien probe having a spacer/linker
agcctaaggtaactatttgtacttctgt

SEQ ID NO: 61 - DNA, 33 bp, Artificial Sequence
  Homo sapien probe having a spacer/linker
agcctctatctgtacttataaggtaactatttg

SEQ ID NO: 62 - DNA, 32 bp, Artificial Sequence
  Homo sapien probe having a spacer/linker
agcctactgtcgagatcctgaaacaattaaac

SEQ ID NO: 63 - DNA, 26 bp, Artificial Sequence
  Homo sapien probe having a spacer/linker
agcctactgtgcctgattcgagatcc

SEQ ID NO: 64 - DNA, 27 bp, Artificial Sequence
  Homo sapien probe having a spacer/linker
agcctactgtcgagagctggagttgga

SEQ ID NO: 65 - DNA, 25 bp, Artificial Sequence
  Homo sapien probe having a spacer/linker
agcctactgtggttcagcgagagct

SEQ ID NO: 66 - DNA, 37 bp, Artificial Sequence
  Homo sapien probe having a spacer/linker
agcctactgtaagtatgttttttttaaagaagaaacg

SEQ ID NO: 67 - DNA, 32 bp, Artificial Sequence
  Homo sapien probe having a spacer/linker
agcctactgtaagactcagagaagtatgtttt

SEQ ID NO: 68 - DNA, 33 bp, Artificial Sequence
  Homo sapien probe having a spacer/linker
agcctactgtaaggtaactatttgtacttctgt

SEQ ID NO: 69 - DNA, 38 bp, Artificial Sequence
  Homo sapien probe having a spacer/linker
agcctactgtctatctgtacttataaggtaactatttg

SEQ ID NO: 70 - DNA, 37 bp, Artificial Sequence
  Homo sapien probe having a spacer/linker
agcctactgatgagtcgagatcctgaaacaattaaac

SEQ ID NO: 71 - DNA, 31 bp, Artificial Sequence
  Homo sapien probe having a spacer/linker
agcctactgatgagtgcctgattcgagatcc

SEQ ID NO: 72 - DNA, 32 bp, Artificial Sequence
  Homo sapien probe having a spacer/linker
aagcctactgatgagtcgagagctggagttgg

SEQ ID NO: 73 - DNA, 30 bp, Artificial Sequence
  Homo sapien probe having a spacer/linker
agcctactgatgagtggttcagcgagagct

SEQ ID NO: 74 - DNA, 42 bp, Artificial Sequence
  Homo sapien probe having a spacer/linker
agcctactgatgagtaagtatgttttttttaaagaagaaacg

SEQ ID NO: 75 - DNA, 37 bp, Artificial Sequence
  Homo sapien probe having a spacer/linker
agcctactgatgagtaagactcagagaagtatgtttt

SEQ ID NO: 76 - DNA, 38 bp, Artificial Sequence
  Homo sapien probe having a spacer/linker
agcctactgatgagtaaggtaactatttgtacttctgt

SEQ ID NO: 77 - DNA, 43 bp, Artificial Sequence
  Homo sapien probe having a spacer/linker
agcctactgatgagtctatctgtacttataaggtaactatttg

SEQ ID NO: 78 - DNA, 6 bp, Artificial Sequence
  Optional sequence on primer for PCR
gcatcc